(19) United States
(12) Patent Application Publication
(10) Pub. No.: US 2025/0121099 A1
(43) Pub. Date: Apr. 17, 2025

(54) CLOSO-DODECAIODODECABORATE COMPLEXES AND METHODS OF USE FOR SAME

(71) Applicant: The Regents of the University of California, Oakland, CA (US)

(72) Inventors: Sophia E. Hollow, Santa Cruz, CA (US); Timothy C. Johnstone, Santa Cruz, CA (US)

(21) Appl. No.: 18/716,761

(22) PCT Filed: Dec. 21, 2022
(86) PCT No.: PCT/US2022/053685

§ 371 Date: Jun. 5, 2024

Related U.S. Application Data
(60) Provisional application No. 63/296,662, filed on Jan. 5, 2022.
(60) Provisional application No. 63/329,695, filed on Apr. 11, 2022.

(51) Int. Cl. A61K 49/04; A61K 47/69

(52) U.S. Cl. CPC A61K 49/0438; A61K 47/6951

(57) ABSTRACT

Substituted cyclodextrin complexes of closo-dodecaiodododecaborate and salts thereof are provided. Complexes according to certain embodiments are soluble in water sufficient to be used as an X-ray contrast agent. X-ray contrast agent compositions and methods for administering the complexes of closo-dodecaiodododecaborate are also described.

### INTRODUCTION

Although recent developments in magnetic resonance and nuclear imaging are driving advancement in medical diagnosis, X-ray imaging remains the most common form of medical imaging, allowing for rapid, non-invasive diagnosis. X-ray tubes used in medical imaging typically contain a tungsten anode and, when operated between 50 and 150 kV, emit radiation with wavelengths ranging from 50 to 9 nm. Radiographic contrast depends on the differences in the extent to which the imaged materials absorb the X-rays used (i.e., radiopacity), which in turn generally scales with the atomic numbers of the elements in those materials. As a result, Ca-containing bone material contrasts well with soft tissues that predominantly contain carbon, hydrogen, nitrogen and oxygen. Contrast between soft tissues can be obtained by introducing an X-ray contrast agent (XCA) of greater or lesser radiopacity. An example of the former is the use of suspended BaSO₄ to image the gastrointestinal tract and an example of the latter is the injection of air into a joint to visualize the articular space. In many cases, e.g., when blockage or pressure is a concern, a soluble XCA is used. The most common soluble XCAs feature iodoarene rings functionalized with water-solubilizing groups. Although these iodinated species are generally well tolerated, some individuals reportedly suffer from contrast-induced nephropathy or disruption of thyroid function.

### SUMMARY

Substituted cyclodextrin complexes of closo-dodecaiodododecaborate and salts thereof are provided. Complexes according to certain embodiments are soluble in water sufficient to be used as an X-ray contrast agent. X-ray contrast agent compositions and methods for administering the complexes of closo-dodecaiodododecaborate are also described.

In some embodiments, complexes of interest include a substituted cyclodextrin which is substituted with one or more groups such as alkoxy, amine, cyano, thiol, halogen, alkyl, substituted alkyl, haloalkyl, heteroalkyl and substituted heteroalkyl. In some instances, the substituted cyclodextrin is a substituted with a hydroxy-substituted alkyl such as a hydroxy-substituted C(1-6) alkyl. In certain embodiments, the substituted cyclodextrin is a 2-hydroxypropyl cyclodextrin. In some instances, the cyclodextrin is selected from an alpha-cyclodextrin (α-CD), a beta-cyclodextrin (β-CD) or a gamma cyclodextrin (γ-CD). In certain instances, the substituted cyclodextrin is 2-hydroxypropyl γ-cyclodextrin. In certain instances, the cyclodextrin is a compound of Formula I:

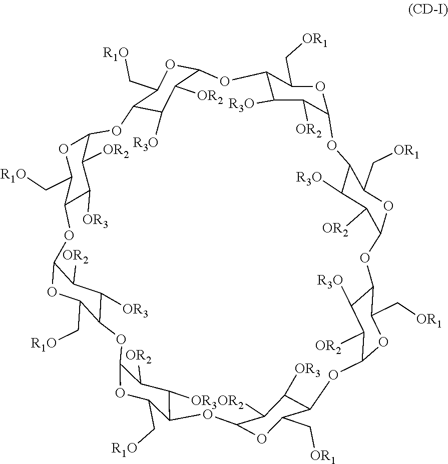

where each R₁, R₂ and R₃ are independently selected from substituted alkyl and substituted heteroalkyl. In some embodiments, any one of R₁, R₂ and R₃ may independently be CH₂CH(OH)CH₃. In certain embodiments, the cyclodextrin is a compound of Formula CD-101

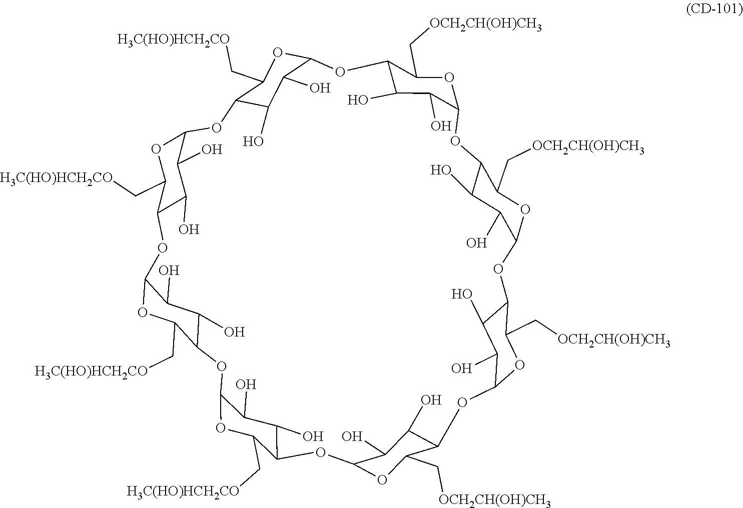

In some embodiments, complexes of interest include a salt of closo-dodecaiodododecaborate. In some instances, the complex includes a ratio of substituted cyclodextrin to closo-dodecaiodododecaborate of from 1:1 to 5:1, such as 2:1, such as 3:1 and including substituted cyclodextrin to closo-dodecaiodododecaborate 4:1. In other instances, the complex includes a ratio of substituted cyclodextrin to closo-dodecaiodododecaborate of from 1:1 to 1:5, such as 1:2, such as 1:3 and including substituted cyclodextrin to closo-dodecaiodododecaborate of 1:4.

Aspects of the present disclosure also include compositions having a substituted cyclodextrin complex of closo-dodecaiodododecaborate. In some embodiments, the composition includes a pharmaceutically acceptable excipient. In some embodiments, the composition includes a buffer. In certain instances, the buffer is a phosphate buffer. In certain embodiments, the composition is formulated for use as an X-ray contrast agent.

Aspects of the disclosure also include methods for administering a substituted cyclodextrin complex of closo-dodecaiodododecaborate to a subject, such as an X-ray contrast agent. In certain embodiments, methods include administering the subject a composition having a substituted cyclodextrin complex of closo-dodecaiodododecaborate and imaging the subject with a source of X-ray radiation. In some instances, the composition is administered orally to the subject. In some instances, the composition is administered to the subject by injection. In some instances, the composition is intravenously administered to the subject. In embodiments, the complex is administered to the subject in an amount sufficient to not cause hemolysis in the subject. In certain embodiments, methods include administering the subject a composition having a substituted cyclodextrin complex of closo-dodecaiodododecaborate and generating an X-ray image of the subject.

### BRIEF DESCRIPTION OF THE FIGURES

FIG. 1 depicts the chemical structures of closo-dodecaiodododecaborate sodium and iodinated X-ray contrast agents iohexol and iodiaxnol.

FIG. 2 depicts stacked ¹¹B NMR spectra (160 MHz) of Na₂B₁₂I₁₂ in a) 10:1 PBS:D₂O after heating at 100° C. for 1 h; b) RBC suspension with 9% D₂O after incubating at room temperature for 24 hours; c) defibrinated bovine blood with 9% D₂O after incubating at room temperature for 24 hours; d) bovine serum with 9% D₂O after incubating at room temperature for 24 hours. Spectra b-d are broadened from sample viscosity and paramagnetic impurities, but no additional signals appear.

FIG. 3 depicts hemolytic activity of Na₂B₁₂I₁₂ according to certain embodiments. FIG. 3A) Hemoglobin (Hb) release from red blood cells (RBCs) suspended in PBS (pH 7.4) containing increasing concentrations of Na₂B₁₂I₁₂. FIG. 3B) Proportion of RBCs lysed by Na₂B₁₂I₁₂ on the basis of the absorption of the supernatant at 413 nm (Soret band) after pelleting. Error bars reflect ±SEM for three independent replicates and the fitted curve was obtained by logistic regression (IC50=66.33 mM).

FIG. 4 depicts ¹H NMR spectrum (d6-DMSO, 500 MHz) of dissolved of Na₂B₁₂I₁₂/γ-CD crystals.

FIG. 5 depicts ¹¹B NMR spectrum (d6-DMSO, 160 MHz) of a solution of dissolved Na₂B₁₂I₁₂/γ-CD crystals.

FIG. 6 depicts the structural interaction of Na₂B₁₂I₁₂ and γ-CD according to certain embodiments. FIG. 6A) X-ray diffraction (Cu Kα) from a crystal containing 7-CD, Na₂B₁₂I₁₂, and DMF showing clean, but low-resolution reflections. Circles are drawn at resolution levels of 3.66, 2.15, and 1.71 Å. FIG. 6B) Semi-empirically (PM6) optimized structure of a putative [(γ-CD)₂(B₁₂I₁₂)]²⁻ complex based on cumulative crystallographic data and previous work with B₁₂Br₁₂²⁻. B₁₂I₁₂²⁻ is shown as spheres and γ-CD as sticks. Color code: I purple, B pink, C green, O red. FIG. 6C) Thermal ellipsoid plot (50% probability level) of Na₂B₁₂I₁₂·6DMF H₂O. Color code: B pink, I purple, Na teal, N blue, 0 red, C grey, H white spheres of arbitrary radius.

FIG. 7 depicts computationally optimized structures of γ-CD complexing B₁₂I₁₂₂. Semi-empirically (PM6) optimized structures of (FIG. 7A) [(γ-CD)(B₁₂I₁₂)]²⁻ and (FIG. 7B) [(HP-γ-CD)(B₁₂I₁₂)]²⁻ highlighting that the 2-hydroxypropyl groups in the latter do not impact the binding of B₁₂I₁₂²⁻. Note that the HP-γ-CD used in this work featured an average of between four and five 2-hydroxypropyl groups. The calculations were performed with four HP groups on alternating glucose units. B₁₂I₁₂²⁻ is shown as spheres and γ-CD as sticks. Color code: I purple, B pink, C green, O red.

FIG. 8 depicts stacked ¹¹B NMR spectra (10:1 PBS:D₂O, 160 MHz) of mixtures of Na₂B₁₂I₁₂ and HP-γ-CD with a combined concentration of 10 mM. Indicated next to each trace is the mole fraction of Na₂B₁₂I₁₂. Dotted line marks the position of the unperturbed Na₂B₁₂I₁₂.

FIG. 9 depicts the Job plot for the interaction of Na₂B₁₂I₁₂ and HP-γ-CD based on the NMR spectra in FIG. 8.

FIG. 10 depicts Hb release from RBCs suspended in PBS (pH 7.4) containing 100 mM Na₂B₁₂I₁₂ with addition of increasing amounts of HP-γ-CD.

FIG. 11 depicts Hb release from RBCs suspended for extended periods (4 h or 24 h) in PBS (pH 7.4) containing 100 mM Na₂B₁₂I₁₂ and 50 mM HP-γ-CD. Hb release within 10 s in the absence of HP-γ-CD (reproduced from FIG. 10) included for reference.

### SELECT DEFINITIONS

The following terms have the following meaning unless otherwise indicated. Any undefined terms have their art recognized meanings.

As used herein, the term “alkyl” by itself or as part of another substituent refers to a saturated branched or straight-chain monovalent hydrocarbon radical derived by the removal of one hydrogen atom from a single carbon atom of a parent alkane. Typical alkyl groups include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl. In some embodiments, an alkyl group comprises from 1 to 20 carbon atoms. In other embodiments, an alkyl group comprises from 1 to 10 carbon atoms. In still other embodiments, an alkyl group comprises from 1 to 6 carbon atoms, such as from 1 to 4 carbon atoms.

“Alkanyl” by itself or as part of another substituent refers to a saturated branched, straight-chain or cyclic alkyl radical derived by the removal of one hydrogen atom from a single carbon atom of an alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkylene” refers to a branched or unbranched saturated hydrocarbon chain, usually having from 1 to 40 carbon atoms, more usually 1 to 10 carbon atoms and even more usually 1 to 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkenyl” by itself or as part of another substituent refers to an unsaturated branched, straight-chain or cyclic alkyl radical having at least one carbon-carbon double bond derived by the removal of one hydrogen atom from a single carbon atom of an alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of an alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyl” by itself or as part of another substituent refers to a radical —C(O)R³⁰, where R³¹ is hydrogen, alkyl, cycloalkyl, cycloheteroalkyl, aryl, arylalkyl, heteroalkyl, heteroaryl, heteroarylalkyl as defined herein and substituted versions thereof. Representative examples include, but are not limited to formyl, acetyl, cyclohexylcarbonyl, cyclohexylmethylcarbonyl, benzoyl, benzylcarbonyl, piperonyl, succinyl, and malonyl, and the like.

The term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Alkoxy” by itself or as part of another substituent refers to a radical —OR³¹ where R³¹ represents an alkyl or cycloalkyl group as defined herein. Representative examples include, but are not limited to, methoxy, ethoxy, propoxy, butoxy, cyclohexyloxy and the like.

“Alkoxycarbonyl” by itself or as part of another substituent refers to a radical —C(O)OR³¹ where R³¹ represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

“Aryl” by itself or as part of another substituent refers to a monovalent aromatic hydrocarbon radical derived by the removal of one hydrogen atom from a single carbon atom of an aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group comprises from 6 to 20 carbon atoms. In certain embodiments, an aryl group comprises from 6 to 12 carbon atoms. Examples of an aryl group are phenyl and naphthyl.

“Arylalkyl” by itself or as part of another substituent refers to an acyclic alkyl radical in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In certain embodiments, an arylalkyl group is (C₇-C₃₀) arylalkyl, e.g., the alkanyl, alkenyl or alkynyl moiety of the arylalkyl group is (C₁-C₁₀) and the aryl moiety is (C₆-C₂₀). In certain embodiments, an arylalkyl group is (C₇-C₂₀) arylalkyl, e.g., the alkanyl, alkenyl or alkynyl moiety of the arylalkyl group is (C₁-C₈) and the aryl moiety is (C₆-C₁₂).

“Arylaryl” by itself or as part of another substituent, refers to a monovalent hydrocarbon group derived by the removal of one hydrogen atom from a single carbon atom of a ring system in which two or more identical or non-identical aromatic ring systems are joined directly together by a single bond, where the number of such direct ring junctions is one less than the number of aromatic ring systems involved. Typical arylaryl groups include, but are not limited to, biphenyl, triphenyl, phenyl-napthyl, binaphthyl, biphenyl-napthyl, and the like. When the numbers of carbon atoms in an arylaryl group are specified, the numbers refer to the carbon atoms comprising each aromatic ring. For example, (C₅-C₁₄) arylaryl is an arylaryl group in which each aromatic ring comprises from 5 to 14 carbons, e.g., biphenyl, triphenyl, binaphthyl, phenylnapthyl, etc. In certain embodiments, each aromatic ring system of an arylaryl group is independently a (C₅-C₁₄) aromatic. In certain embodiments, each aromatic ring system of an arylaryl group is independently a (C₅-C₁₀) aromatic. In certain embodiments, each aromatic ring system is identical, e.g., biphenyl, triphenyl, binaphthyl, trinaphthyl, etc.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane and the like. In certain embodiments, the cycloalkyl group is (C₃-C₁₀) cycloalkyl. In certain embodiments, the cycloalkyl group is (C₃-C₇) cycloalkyl.

“Cycloheteroalkyl” or “heterocyclyl” by itself or as part of another substituent, refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, —S—, —S—S—, —O—S—, —NR³⁷R³⁸—, ═N—N═, —N═N—, —N═N—NR³⁹R⁴⁰, —PR⁴¹—, —P(O)₂—, —POR⁴²—, —O—P(O)₂—, —S—O—, —S—(O)—, —SO₂—, —SnR⁴³R⁴⁴— and the like, where R³⁷, R³⁸, R³⁹, R⁴⁰, R⁴¹, R⁴², R⁴³ and R⁴⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent, refers to a monovalent heteroaromatic radical derived by the removal of one hydrogen atom from a single atom of a heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, benzodioxole and the like. In certain embodiments, the heteroaryl group is from 5-20 membered heteroaryl. In certain embodiments, the heteroaryl group is from 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent, refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In certain embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl. In certain embodiments, the heteroarylalkyl group is 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“Aromatic Ring System” by itself or as part of another substituent, refers to an unsaturated cyclic or polycyclic ring system having a conjugated π electron system. Specifically included within the definition of “aromatic ring system” are fused ring systems in which one or more of the rings are aromatic and one or more of the rings are saturated or unsaturated, such as, for example, fluorene, indane, indene, phenalene, etc. Typical aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Heteroaromatic Ring System” by itself or as part of another substituent, refers to an aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical heteroaromatic ring systems include, but are not limited to, arsindole, carbazole, 3-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Substituted” refers to a group in which one or more hydrogen atoms are independently replaced with the same or different substituent(s). Typical substituents include, but are not limited to, alkylenedioxy (such as methylenedioxy), -M, —R⁶⁰, —O—, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶¹OR⁶¹, —NR⁶²C(S)NR⁶OR⁶¹, —NR⁶²C(NR⁶³)NR⁶OR⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. In certain embodiments, substituents include -M, —R⁶⁰, ═O, —OR⁶⁰, —SR⁶, —S⁻, ═S, —NR⁶⁰R⁶¹, ═NR⁶, —CF₃, —CN, —OCN, —SCN, —NO, —NO₂, ═N₂, —N₃, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —NR⁶²C(O)NR⁶⁰R⁶¹. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O⁻), —OP(O)(OR⁶⁰)(OR⁶¹), —C(O)R⁶⁰, —C(O)OR⁶⁰, —C(O)NR⁶⁰R⁶¹, —C(O)O⁻. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)OR⁶⁰, —C(O)O⁻, where R⁶⁰, R⁶¹ and R⁶² are as defined above. For example, a substituted group may bear a methylenedioxy substituent or one, two, or three substituents selected from a halogen atom, a (1-4C)alkyl group and a (1-4C)alkoxy group.

“Pharmaceutically acceptable carrier” refers to a diluent, adjuvant, excipient or vehicle with, or in which a compound is administered.

### DETAILED DESCRIPTION

Substituted cyclodextrin complexes of closo-dodecaiodododecaborate and salts thereof are provided. Complexes according to certain embodiments are soluble in water sufficient to be used as an X-ray contrast agent. X-ray contrast agent compositions and methods for administering the complexes of closo-dodecaiodododecaborate are also described.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the compounds and methods have or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterised, and tested for biological activity). In addition, all sub-combinations of the chemical groups listed in the embodiments describing such variables are also specifically embraced by the present invention and are disclosed herein just as if each and every such sub-combination of chemical groups was individually and explicitly disclosed herein.

Reference will now be made in detail to various embodiments. It will be understood that the invention is not limited to these embodiments. To the contrary, it is intended to cover alternatives, modifications, and equivalents as may be included within the spirit and scope of the allowed claims.

Substituted γ-Cyclodextrin Complexes of closo-dodecaiodododecaborate and Compositions Thereof In embodiments, complexes of the closo-dodecaiodododecaborate include a substituted cyclodextrin which is substituted, partially or completely (e.g., at the 2, 3, or 6 positions) with one or more groups such as hydroxy, alkoxy, amine, cyano, thiol, halogen, alkyl, substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl or a salt, solvate or hydrate thereof.

In embodiments, closo-dodecaiodododecaborate refers to B₁₂I₁₂²⁻, shown below:

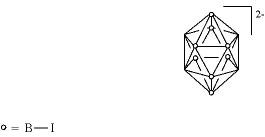

In embodiments, “salts” of the compounds of the present disclosure may include: (1) salts formed when the charge of the anion is balanced by a metal ion, e.g., an alkali metal ion, an alkaline earth ion, or an aluminum ion; or (2) salts formed when the charge of the anion is balanced by an organic cation, e.g., a tetraalkyl ammonium ion, a trialkylammonium ion, or a tetraarylphosphonium ion. In certain embodiments, complexes include a sodium salt of closo-dodecaiodododecaborate.

The term “solvate” as used herein refers to a complex or aggregate formed by one or more molecules of a solute, e.g. a compound of Formula (I) or a salt thereof, and one or more molecules of a solvent. Such solvates may be crystalline solids having a substantially fixed molar ratio of solute and solvent. Representative solvents include by way of example, water, methanol, ethanol, isopropanol, acetic acid, and the like. When the solvent is water, the solvate formed is a hydrate.

In some embodiments, the substituted cyclodextrin is a substituted with a hydroxy-substituted alkyl. In some instances, hydroxy-substituted alkyl is a C(1-6)alkyl. In some instances, the alkyl group of the hydroxy-substituted alkyl is selected from methyl, ethyl, n-propyl, isopropyl, n-butyl, t-butyl, pentyl and hexyl. In certain instances, the substituted cyclodextrin is a 2-hydroxypropyl cyclodextrin.

In some instances, the cyclodextrin is selected from an alpha-cyclodextrin (α-CD), a beta-cyclodextrin (β-CD) or a gamma-cyclodextrin (γ-CD). In certain instances, the substituted cyclodextrin is 2-hydroxypropyl-γ-cyclodextrin. In certain instances, the cyclodextrin is a compound of Formula I:

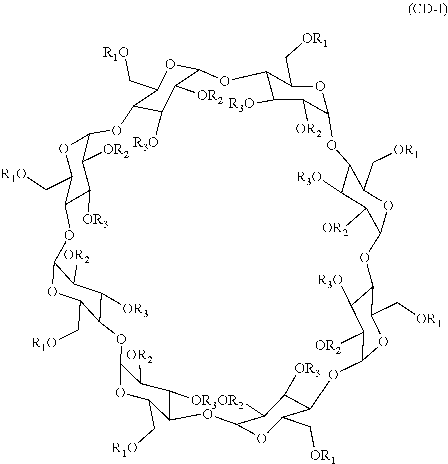

where each R₁, R₂ and R₃ are independently selected from substituted alkyl and substituted heteroalkyl. In some embodiments, any one of R₁, R₂ and R₃ may independently be CH₂CH(OH)CH₃. In certain embodiments, the cyclodextrin is a compound of Formula CD-101

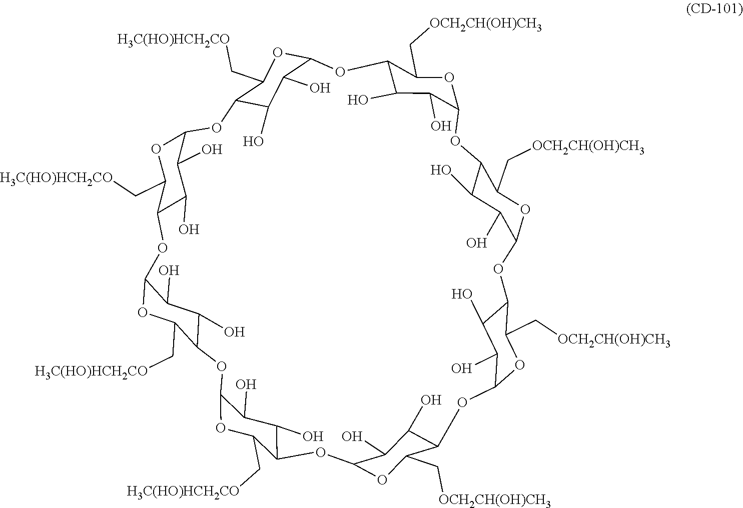

In some instances, the complex includes a ratio of substituted cyclodextrin to closo-dodecaiodododecaborate of from 1:1 to 5:1, such as 2:1, such as 3:1 and including substituted cyclodextrin to closo-dodecaiodododecaborate 4:1. In other instances, the complex includes a ratio of substituted cyclodextrin to closo-dodecaiodododecaborate of from 1:1 to 1:5, such as 1:2, such as 1:3 and including substituted cyclodextrin to closo-dodecaiodododecaborate of 1:4.

Aspects of the present disclosure also include compositions having a pharmaceutically acceptable carrier and one or more of the complexes described above. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc. For example, the one or more excipients may include sucrose, starch, mannitol, sorbitol, lactose, glucose, cellulose, talc, calcium phosphate or calcium carbonate, a binder (e.g., cellulose, methylcellulose, hydroxymethylcellulose, polypropylpyrrolidone, polyvinylpyrrolidone, gelatin, gum arabic, poly(ethylene glycol), sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

The closo-dodecaiodododecaborate substituted cyclodextrin complexes may be formulated into compositions (e.g., X-ray contrast agent) by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. In certain embodiments, the conjugate compounds are formulated for injection. For example, compositions of interest may be formulated for intravenous or intraperitoneal administration.

In pharmaceutical dosage forms, the compound may be administered in the form of its pharmaceutically acceptable salts, or it may also be used alone or in appropriate association, as well as in combination, with other pharmaceutically active compounds. The following methods and excipients are merely exemplary and are in no way limiting.

In some embodiments, compositions of interest include an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. In some instances, compositions of interst further include a preservative. Suitable preservatives include, but are not limited to, a benzyl alcohol, phenol, chlorobutanol, benzalkonium chloride, and the like. In many cases, the composition is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

In some embodiments, compositions include other additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Where the composition is formulated for injection, the compounds may be formulated by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The closo-dodecaiodododecaborate substituted cyclodextrin complexes may be present in the composition in an amount of from 0.0001 mg to about 5000 mg, e.g., from about 1 mg to about 25 mg, from about 25 mg to about 50 mg, from about 50 mg to about 100 mg, from about 100 mg to about 200 mg, from about 200 mg to about 250 mg, from about 250 mg to about 500 mg, from about 500 mg to about 1000 mg, or from about 1000 mg to about 5000 mg. In some instances, the closo-dodecaiodododecaborate is present in the composition at a concentration that is sufficiently high for use as an X-ray contrast agent. In some instance, the concentration of the closo-dodecaiodododecaborate is from 10 mM to 1000 mM, such as from 20 mM to 900 mM, such as from 30 mM to 800 mM, such as from 40 mM to 700 mM, such as from 50 mM to 600 mM, such as from 60 mM to 500 mM, such as from 70 mM to 400 mM, such as from 80 mM to 300 mM and including from 100 mM to 200 mM. In some embodiments, the closo-dodecaiodododecaborate provides for a composition having an iodine concentration of 10 mg/mL or more, such as 15 mg/mL or more, such as 20 mg/mL or more, such as 25 mg/mL or more, such as 50 mg/mL or more, such as 100 mg/mL or more, such as 200 mg/mL or more, such as 250 mg/mL or more, such as 300 mg/mL or more, such as 350 mg/mL or more, such as 400 mg/mL or more, such as 450 mg/mL or more, such as 500 mg/mL or more, such as 550 mg/mL or more, such as 600 mg/mL or more, such as 650 mg/mL or more, such as 700 mg/mL or more, such as 750 mg/mL or more, such as 800 mg/mL or more, such as 850 mg/mL or more, such as 900 mg/mL or more, such as 950 mg/mL or more and including 1000 mg/mL or more.

The substituted cyclodextrin may be present in the composition in an amount of from 0.01 equivalents or more to the closo-dodecaiodododecaborate, such as 0.05 equivalents or more, such as 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more and including present in an amount of 1.0 equivalents or more to the closo-dodecaiodododecaborate.

Methods for Using Complexes of closo-Dodecaiodododecaborate and Substituted γ-Cyclodextrin

As summarized above, aspects of the present disclosure also include administering the substituted cyclodextrin complexes of closo-dodecaiodododecaborate to a subject. In certain embodiments, compositions of substituted cyclodextrin complexes of closo-dodecaiodododecaborate administered to the subject are formulated as an X-ray contrast agent. In practicing the subject methods, an effective amount of one or more of the complexes disclosed herein is administered to a subject. In embodiments, the term “subject” is meant the person or organism to which the compound is administered. As such, subjects of the present disclosure may include but are not limited to mammals, e.g., humans and other primates, such as chimpanzees and other apes and monkey species, dogs, rabbits, cats and other domesticated pets; and the like, where in certain embodiments the subject are humans. The term “subject” is also meant to include a person or organism of any age, weight or other physical characteristic, where the subjects may be an adult, a child, an infant or a newborn.

Complexes and compositions as described herein may be administered to a subject by any convenient protocol, including, but not limited, to intraperitoneally, topically, orally, sublingually, parenterally, intravenously, vaginally, rectally as well as by transdermal protocols. In certain embodiments, the subject compounds are administered by intravenous injection. In certain embodiments, the subject compounds are administered by intraperitoneal injection.

In some instances, the substituted cyclodextrin complexes of closo-dodecaiodododecaborate are administered in an amount of from 0.0001 mg to about 5000 mg, e.g., from about 1 mg to about 25 mg, from about 25 mg to about 50 mg, from about 50 mg to about 100 mg, from about 100 mg to about 200 mg, from about 200 mg to about 250 mg, from about 250 mg to about 500 mg, from about 500 mg to about 1000 mg, or from about 1000 mg to about 5000 mg. In some instances, the closo-dodecaiodododecaborate is administered at a concentration of from 10 mM to 1000 mM, such as from 20 mM to 900 mM, such as from 30 mM to 800 mM, such as from 40 mM to 700 mM, such as from 50 mM to 600 mM, such as from 60 mM to 500 mM, such as from 70 mM to 400 mM, such as from 80 mM to 300 mM and including from 100 mM to 200 mM. In some embodiments, the closo-dodecaiodododecaborate is administered in an amount to provide an iodine concentration of 10 mg/mL or more, such as 15 mg/mL or more, such as 20 mg/mL or more, such as 25 mg/mL or more, such as 50 mg/mL or more, such as 100 mg/mL or more, such as 200 mg/mL or more, such as 250 mg/mL or more, such as 300 mg/mL or more, such as 350 mg/mL or more, such as 400 mg/mL or more, such as 450 mg/mL or more, such as 500 mg/mL or more, such as 550 mg/mL or more, such as 600 mg/mL or more, such as 650 mg/mL or more, such as 700 mg/mL or more, such as 750 mg/mL or more, such as 800 mg/mL or more, such as 850 mg/mL or more, such as 900 mg/mL or more, such as 950 mg/mL or more and including 1000 mg/mL or more.

The amount of substituted cyclodextrin complexes of closo-dodecaiodododecaborate administered to the subject may vary, such as ranging from about 0.0001 mg/day to about 10,000 mg/day, such as from about 0.001 mg/day to about 9000 mg/day, such as from 0.01 mg/day to about 8000 mg/day, such as from about 0.1 mg/day to about 7000 mg/day, such as from about 1 mg/day to about 6000 mg/day, including from about 5 mg/day to about 5000 mg/day. Each dosage of the compound or pharmaceutically acceptable salt administered to the subject may vary ranging from about 1 mg/kg to about 1000 mg/kg, such as from about 2 mg/kg to about 900 mg/kg, such as from about 3 mg/kg to about 800 mg/kg, such as from about 4 mg/kg to about 700 mg/kg, such as from 5 mg/kg to about 600 mg/kg, such as from 6 mg/kg to about 500 mg/kg, such as from 7 mg/kg to about 400 mg/kg, such as from about 8 mg/kg to about 300 mg/kg, such as from about 9 mg/kg to about 200 mg/kg and including from about 10 mg/kg to about 100 mg/kg. In certain embodiments, protocols may include multiple dosage intervals. By “multiple dosage intervals” is meant that two or more dosages of the compound is administered to the subject in a sequential manner. In practicing methods of the present disclosure, regimens may include two or more dosage intervals, such as three or more dosage intervals, such as four or more dosage intervals, such as five or more dosage intervals, including ten or more dosage intervals. The duration between dosage intervals in a multiple dosage interval protocol may vary, depending on the physiology of the subject or by the protocol as determined by a health care professional. For example, the duration between dosage intervals in a multiple dosage protocol may be predetermined and follow at regular intervals. As such, the time between dosage intervals may vary and may be 1 day or longer, such as 2 days or longer, such as 4 days or longer, such as 6 days or longer, such as 8 days or longer, such as 12 days or longer, such as 16 days or longer and including 24 days or longer. In certain embodiments, multiple dosage interval protocols provide for a time between dosage intervals of 1 week or longer, such as 2 weeks or longer, such as 3 weeks or longer, such as 4 weeks or longer, such as 5 weeks or longer, including 6 weeks or longer.

In certain embodiments, methods include imaging the subject with a source of X-ray radiation. In certain instances, an X-ray image (e.g., a radiograph) is generated. The subject may be exposed to the X-ray radiation to image the subject according to any convenient radiology protocol, such as determined by a qualified health care professional. In some instances, the subject is imaged with the source of X-ray radiation 1 minute or more after administering the substituted cyclodextrin complexes of closo-dodecaiodododecaborate to the subject, such as after 5 minutes or more, such as after 10 minutes or more, such as after 15 minutes or more, such as after 30 minutes or more, such as after 45 minutes or more, such as after 1 hour or more and including 2 hours or more after administering the substituted cyclodextrin complexes of closo-dodecaiodododecaborate to the subject.

Aspects, including embodiments, of the subject matter described herein may be beneficial alone or in combination, with one or more other aspects or embodiments. Without limiting the description, certain non-limiting aspects of the disclosure numbered 1-40 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A complex comprising:
a substituted cyclodextrin; and
a closo-dodecaiodododecaborate or a salt thereof.
   2. The complex according to 1, wherein the substituted cyclodextrin is substituted with one or more groups selected from alkoxy, amine, cyano, thiol, halogen, alkyl, substituted alkyl, haloalkyl, heteroalkyl and substituted heteroalkyl.
   3. The complex according to 2, wherein the substituted cyclodextrin is substituted with a hydroxy-substituted alkyl.
   4. The complex according to 3, wherein the substituted cyclodextrin comprises 2-hydroxypropyl cyclodextrin.
   5. The complex according to any one of 1-4, wherein the substituted cyclodextrin is a gamma cyclodextrin (γ-CD).
   6. The complex according to any one of 1-4, wherein the substituted cyclodextrin is substituted at the 2 position.
   7. The complex according to any one of 1-4, wherein the substituted cyclodextrin is substituted at the 3 position.
   8. The complex according to any one of 1-4, wherein the substituted cyclodextrin is substituted at the 6 position.
   9. The complex according to 5, wherein the cyclodextrin is a compound of formula CD-I:

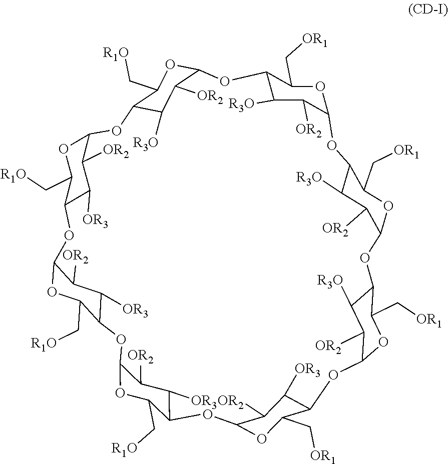

wherein each R₁, R₂ and R₃ are independently selected from substituted alkyl and substituted heteroalkyl.
   10. The complex according to 9, wherein the substituted cyclodextrin is a compound of formula CD-101:

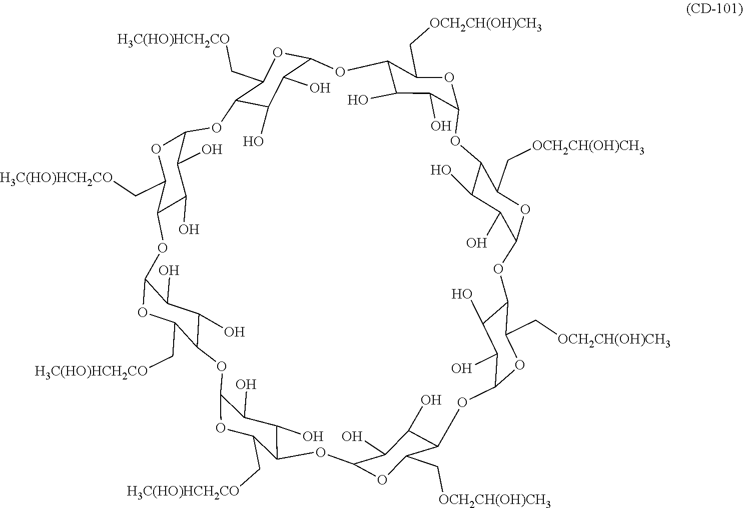

11. The complex according to any one of 1-10, wherein the complex comprises a salt of closo-dodecaiodododecaborate.
12. The complex according to 11, wherein the complex comprises a sodium salt of closo-dodecaiodododecaborate.
13. The complex according to any one of 1-12, wherein the complex comprises a 1:1 ratio of the substituted cyclodextrin to closo-dodecaiodododecaborate.
14. The complex according to any one of 1-12, wherein the complex comprises a 2:1 ratio of the substituted cyclodextrin to closo-dodecaiodododecaborate.
15. A composition comprising:
a complex comprising:
   a substituted cyclodextrin; and
   a closo-dodecaiodododecaborate salt; and
a pharmaceutically acceptable excipient.
   16. The composition according to 15, wherein the composition is formulated for use as an X-ray contrast agent.
   17. The composition according to any one of 15-16, wherein the pharmaceutically acceptable excipient comprises a buffer.
   18. The composition according to 17, wherein the buffer is a phosphate buffer.
   19. The composition according to any one of 15-18, wherein the closo-dodecaiodododecaborate salt is present in the composition in an amount from 5 mM to 125 mM.
   20. The composition according to any one of 15-18, wherein the closo-dodecaiodododecaborate salt is present in the composition in an amount from 20 mM to 100 mM.
   21. The composition according to any one of 15-20, wherein the substituted cyclodextrin is substituted with one or more groups selected from alkoxy, amine, cyano, thiol, halogen, alkyl, substituted alkyl, haloalkyl, heteroalkyl and substituted heteroalkyl.
   22. The composition according to 21, wherein the substituted cyclodextrin is substituted at the 2 position.
   23. The composition according to 21, wherein the substituted cyclodextrin is substituted at the 3 position.
   24. The composition according to 21, wherein the substituted cyclodextrin is substituted at the 6 position.
   25. The composition according to any one of 21-24, wherein the substituted cyclodextrin is substituted with a hydroxy-substituted alkyl.
   26. The composition according to any one of 21-24, wherein the substituted cyclodextrin comprises 2-hydroxypropyl cyclodextrin.
   27. The composition according to any one of 15-26, wherein the substituted cycldextrin is a gamma cyclodextrin (γ-CD).
   28. The composition according to 27, wherein the substituted cyclodextrin is a compound of formula CD-I:

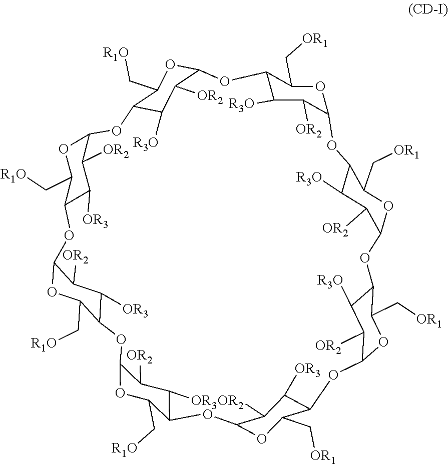

wherein each R₁, R₂ and R₃ are independently selected from substituted alkyl and substituted heteroalkyl.
   29. The composition according to 28, wherein the substituted cyclodextrin is a compound of formula CD-101:

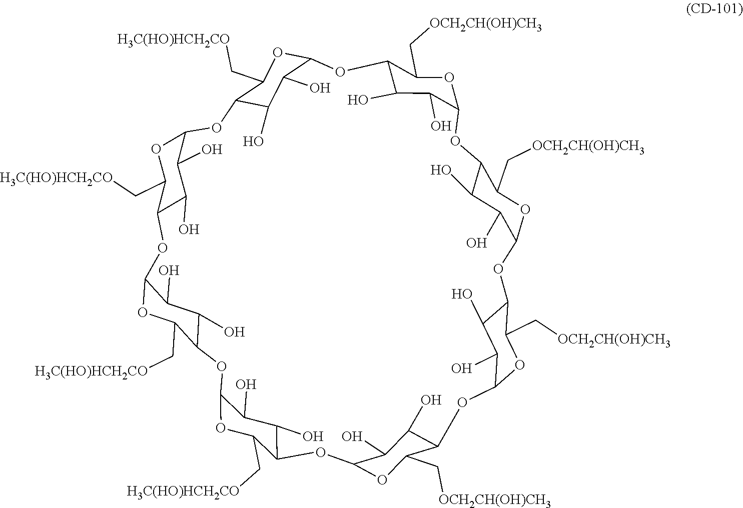

30. The composition according to any one of 15-29, wherein the complex comprises a salt of closo-dodecaiodododecaborate.
31. The composition according to 30, wherein the complex comprises a sodium salt of closo-dodecaiodododecaborate.
32. The composition according to any one of 15-31, wherein the complex comprises a 1:1 ratio of the substituted cyclodextrin to closo-dodecaiodododecaborate.
33. The composition according to any one of 15-31, wherein the complex comprises a 2:1 ratio of the substituted cyclodextrin to closo-dodecaiodododecaborate.
34. A method comprising administering a composition according to any one of claims 15-33 to a subject.
35. The method according to 34, wherein the method comprises imaging the subject with a source of X-ray radiation.
36. The method according to any one of 34-35, wherein the composition is administered orally to the subject.
37. The method according to any one of 34-35, wherein the composition is administered to the subject by injection.
38. The method according to any one of 34-35, wherein the composition is administered intravenously to the subject.
39. The method according to 38, wherein the composition comprises the complex in an amount sufficient that does not cause hemolysis in the subject.
40. The method according to any one of 34-39, wherein the method further comprises generating an X-ray image of the subject.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

### Abstract

Na₂B₁₂I₁₂ has many of the properties desired by an X-ray contrast agent but is lethal at the concentrations needed for medical imaging. PBS solutions with greater than 50 mM Na₂B₁₂I₁₂ induce hemolysis, consistent with the superchaotropic nature of the B₁₂I₁₂ anion. Demonstrated herein are studies where the presence of less than 1 equiv of 2-hydroxypropyl-γ-cyclodextrin prevents hemolysis and provides for use of the B₁₂I₁₂ anion as an X-ray contrast agent.

### Introduction

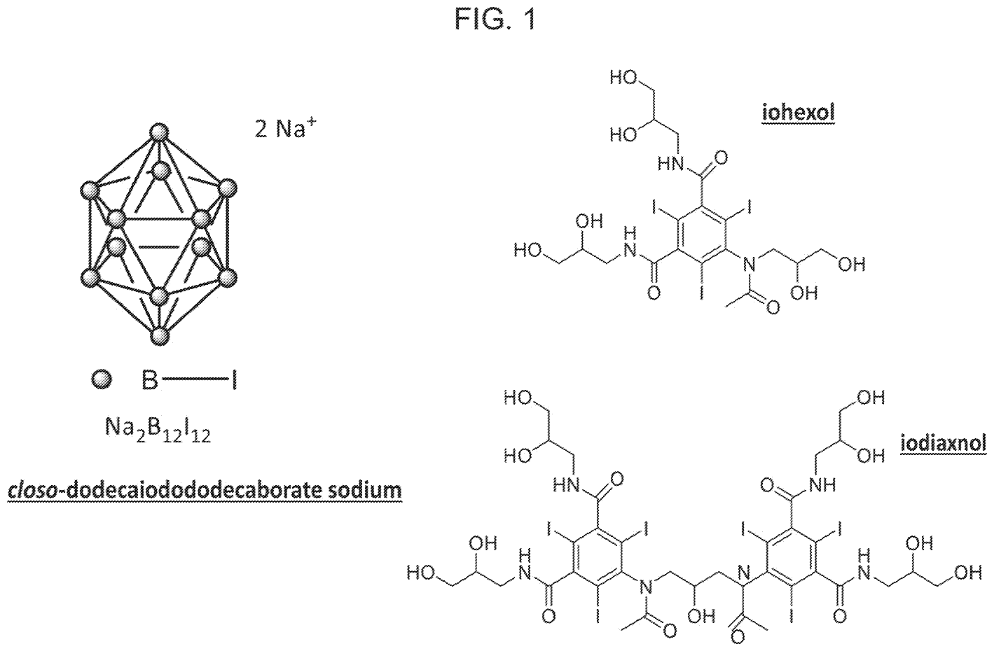

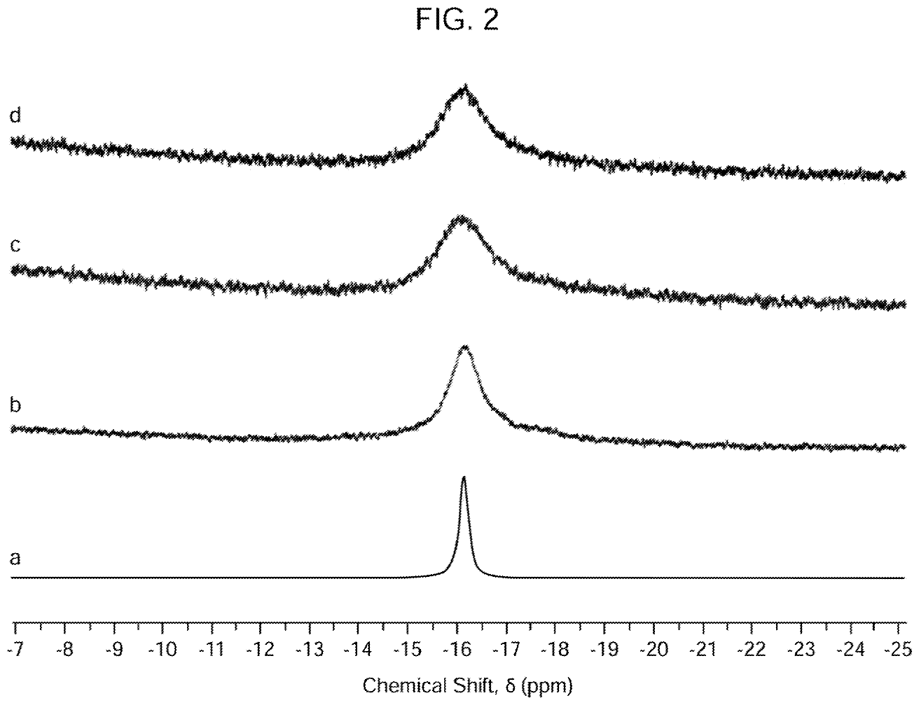

The icosahedral closo-dodecaborate scaffold, which occupies a volume approximately the same as that of an adamantyl group and roughly 50% larger than that of the sphere described by a rotating phenyl ring, provides a rigid and biostable icosahedral framework upon which to construct functional molecules. In the context of XCA design, certain iodine-containing XCAs feature 1,3,5-triiodophenyl groups (see FIG. 1 which compares the structure of icosahedral closo-dodecaborate with iohexol and iodixanol). Although periodinated rings would afford greater contrast, they severely impair solubility. In contrast, periodinated B₁₂I₁₂²⁻ forms salts that are highly water-soluble. For example, Na₂B₁₂I₁₂ is 90% iodine by mass and can be prepared as solutions with >200 mM concentration (>300 mg iodine mL⁻¹). Na₂B₁₂I₁₂ has been shown to be toxic to mice and cats (Ojemann et al., Angiology, 1964, 15, 273-275). While not being bound by theory, the toxicity of Na₂B₁₂I₁₂ is not likely to stem from its chemical reactivity. The B—B and B—I bonds are stable and there is a distinct lack of reactivity of the B₁₂I₁₂²⁻ anion. Indeed, the B₁₂I₁₂²⁻ anion remains unchanged after treatment with Cl₂ gas, heating to 85° C. in 5M NaOH or heating to 150° C. in H₂SO₄. The inertness of the B₁₂I₁₂²⁻ anion under biological conditions is demonstrated by a lack of new ¹¹B NMR signals after refluxing Na₂B₁₂I₁₂ in phosphate-buffered saline (PBS, pH 7.4) for 1 hour. There was also an absence of new signals after 24-hour incubation at room temperature with a suspension of human red blood cells (RBCs), bovine serum, or defibrinated bovine blood (FIG. 2).

The biological effects of the B₁₂I₁₂²⁻ anion may also be determined from inorganic characterizations of the B₁₂X₁₂²⁻ anion where X is fluorine (F), chlorine (Cl), bromine (Br) or iodine (I). B₁₂X₁₂²⁻ anions exhibit chaotropism that far outstrips that of the classical Hofmeister series chaotropes, and are considered to be superchaotropes. One characteristic of this chaotropism is the ability of the B₁₂X₁₂²⁻ anions to enhance the release of compounds complexed with liposomes. Accordingly, at high concentrations of the B₁₂I₁₂²⁻ anion sufficient to be used as an XCA, the superchaotropic activity of the B₁₂I₁₂²⁻ anion disrupts the integrity of cell membranes.

### General Synthetic Procedures

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the compounds of the present disclosure, it may be necessary and/or desirable to protect sensitive or reactive groups on any of the molecules concerned. This can be achieved by means of conventional protecting groups as described in standard works, such as T. W. Greene and P. G. M. Wuts, “Protective Groups in Organic Synthesis”, Fourth edition, Wiley, New York 2006. The protecting groups can be removed at a convenient subsequent stage using methods known from the art.

The compounds described herein can contain one or more chiral centers and/or double bonds and therefore, can exist as stereoisomers, such as double-bond isomers (i.e., geometric isomers), enantiomers or diastereomers. Accordingly, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds can also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that can be incorporated into the compounds disclosed herein include, but are not limited to, ²H, ³H, ¹¹C, ¹³C, ¹⁴C, ¹⁵N, ¹⁸O, ¹⁷O, etc. Compounds can exist in unsolvated forms as well as solvated forms, including hydrated forms. In general, compounds can be hydrated or solvated. Certain compounds can exist in multiple crystalline or amorphous forms. In general, all physical forms are equivalent for the uses contemplated herein and are intended to be within the scope of the present disclosure.

The nomenclature used herein to name the subject compounds is illustrated in the Examples herein. When possible, this nomenclature has generally been derived using the commercially-available AutoNom software (MDL, San Leandro, Calif).

### Materials and Methods

General methods. Na₂B₁₂I₁₂ was obtained from Katchem, γ-cyclodextrin (γ-CD) was obtained from Combi-Blocks, 2-hydroxypropyl-γ-cyclodextrin (HP-γ-CD; 0.6 functionalized, average Mw=1580 Da) was obtained from Sigma-Aldrich, phosphate-buffered saline (PBS) was obtained from Fisher Scientific. The reagents were used as received. D₂O was obtained from Cambridge Isotopes. Washed single-donor human red blood cells (RBCs) were obtained from Innovative Research. These are RBCs that have been separated from type 0 positive whole blood and resuspended in Alsever's Solution. Defibrinated bovine blood was obtained from HemoStat Laboratories. All experiments were performed under ambient conditions. NMR spectra were collected using a Bruker Avance III HD 500 spectrometer equipped with a multinuclear Smart Probe. The frequencies of the ¹H and ¹¹B NMR signals are reported in ppm as chemical shifts from TMS and BF₃·Et₂O, respectively. Electronic absorption spectra were recorded on VWR UV-6300PC double beam spectrophotometer.
Stability of Na₂B₁₂I₁₂: refluxing PBS. A 500 μL aliquot of a 10 mM solution of Na₂B₁₂I₁₂ in PBS (pH 7.4) was placed in an oil bath pre-heated to 100° C. After incubating in this bath for 1 h, the sample was removed, cooled to room temperature, and spiked with 50 μL D₂O prior to acquiring a ¹¹B NMR spectrum.
Stability of Na₂B₁₂I₁₂: human RBCs. A 275 μL aliquot of a 20 mM solution of Na₂B₁₂I₁₂ in PBS (pH 7.4) was added to a 225 μL suspension of human RBCs. The mixture was allowed to stand at room temperature for 24 h. It was then spiked with 50 μL D₂O prior to acquiring a ¹¹B NMR spectrum.
20 Stability of Na₂B₁₂I₁₂: bovine serum. A 1 mL aliquot of defibrinated bovine blood was centrifuged for 15 s at 15,850×g. A 225 μL aliquot of the serum supernatant was combined with a 275 μL aliquot of a 20 mM solution of Na₂B₁₂I₁₂ in PBS (pH 7.4). The mixture was allowed to stand at room temperature for 24 h. It was then spiked with 50 μL D₂O prior to acquiring a ¹¹B NMR spectrum.
Stability of Na₂B₁₂I₁₂: bovine blood. A 225 μL aliquot of defibrinated bovine blood was combined with a 275 μL aliquot of a 20 mM solution of Na₂B₁₂I₁₂ in PBS (pH 7.4). The mixture was allowed to stand at room temperature for 24 h. It was then spiked with 50 μL D₂O prior to acquiring a ¹¹B NMR spectrum.
Job plot of HP-γ-CD and Na₂B₁₂I₁₂. Solutions of Na₂B₁₂I₁₂ (20 mM) and HP-T-CD (20 mM) were prepared in PBS (pH 7.4). Aliquots of these solutions were combined and diluted as needed with PBS to afford 500 μL solutions that varied continuously in concentration of Na₂B₁₂I₁₂ and HP-γ-CD from 0 to 10 mM, under the constraint that their molar concentrations summed to 10 mM. Each sample was spiked with D₂O and analyzed by ¹¹B NMR spectroscopy.
Hemolytic activity of Na₂B₁₂I₁₂. A 200 mM solution of Na₂B₁₂I₁₂ was prepared in PBS (pH 7.4). Aliquots of this solution were diluted to a target concentration of 10-100 mM by dilution with PBS to give a final volume of 250 μL. A 1 mL aliquot of a suspension of human RBCs was pelleted (15 s at 15,850×g), the supernatant was discarded, and the cells were resuspended to a volume of 1 mL with fresh PBS. The cells were pelleted again and were washed a total of three times in this manner and resuspended in a final volume of 1 mL of PBS. A 10 μL aliquot of freshly resuspended RBCs was added to one of the 250 μL solutions of Na₂B₁₂I₁₂. The mixture was vortexed for 10 seconds and then pelleted by centrifugation at 15,850×g for 10 seconds. A 200 μL aliquot of the supernatant was removed and diluted with 550 μL of PBS. The absorbance of this solution was measured from 700 to 350 nm. The subsequent samples were then measured in turn. Three independent replicates were obtained for each concentration of hemolytic agents and the data were modeled using logistic regression. The absorbance corresponding to 100% lysis was confirmed by adding a 10 μL aliquot of freshly resuspended RBCs to 250 μL of hemolysis buffer (0.15 M NH₄Cl and 10 mM KHCO₃). The sample was then processed identically to the borate-treated samples.

Hemolytic activity of Na₂B₁₂I₁₂ in the presence of HP-γ-CD. Solutions of Na₂B₁₂I₁₂ (200 mM) and HP-γ-CD (200 mM) were prepared in PBS (pH 7.4). Aliquots of these stock solutions were combined, diluting with PBS, if necessary, to give 250 μL solutions that were 100 mM in Na₂B₁₂H₁₂ and varied in HP-T-CD concentration from 0 to 40 mM. A 1 mL aliquot of a suspension of human RBCs was pelleted (15 s at 15,850×g), the supernatant was discarded, and the cells were resuspended to a volume of 1 mL with fresh PBS. The cells were pelleted again and were washed a total of three times in this manner and resuspended in a final volume of 1 mL of PBS. A 10 μL aliquot of freshly resuspended RBCs was added to one of the 250 μL solutions of Na₂B₁₂I₁₂ with or without HP-γ-CD. The mixture was vortexed for 10 seconds and then pelleted by centrifugation at 15,850×g for 10 seconds. A 200 μL aliquot of the supernatant was removed and diluted with 550 μL of PBS. The absorbance of this solution was measured from 700 nm to 350 nm. The subsequent samples were then measured in turn.

Extended exposure hemolytic activity of Na₂B₁₂I₁₂ in the presence of 0.5 equiv HP-γ-CD. Solutions of Na₂B₁₂I₁₂ (200 mM) and HP-γ-CD (200 mM) were prepared in PBS (pH 7.4). Aliquots of these stock solutions were combined, diluting with PBS, if necessary, to give 250 μL solutions that were 100 mM in Na₂B₁₂H₁₂ and 50 mM in HP-γ-CD. A 1 mL aliquot of a suspension of human RBCs was pelleted (15 seconds at 15,850×g), the supernatant was discarded, and the cells were resuspended to a volume of 1 mL with fresh PBS. The cells were pelleted again and were washed a total of three times in this manner and resuspended in a final volume of 1 mL of PBS. A 10 μL aliquot of freshly resuspended RBCs was added to each of two 250 μL solutions of Na₂B₁₂I₁₂ and HP-γ-CD. The mixtures were vortexed and then incubated at room temperature for either 4 hours or 24 hours. After the prescribed time, the samples were pelleted by centrifugation at 15,850×g for 10 seconds. A 200 μL aliquot of the supernatant was removed and diluted with 550 μL of PBS. The absorbance of this solution was measured from 700 to 350 nm.
Crystallography: Complex of Na₂B₁₂I₁₂ and γ-CD. Crystals were obtained by allowing diethyl ether to diffuse in the vapor phase into a 0.5 mL DMF solution containing Na₂B₁₂I₁₂ (10 mg, 5.9 μmol) and γ-CD (15 mg, 11.8 μmol). Over the course of 3 days, colorless crystals formed. Microscopic analysis of these crystals between crossed polarizers revealed them to remain perpetually extinguished regardless of orientation. A single-crystal sample was coated in Paratone oil and mounted on a MiTeGen polyimide loop and cooled to 100 K on a Rigaku Synergy-S X-ray diffractometer. Diffraction of Cu Kα radiation from a PhotonJet-S microfocus source was detected using a HyPix-6000HE hybrid photon counting detector, but reflections were only observed to a resolution of 1.5 Å and a satisfactory solution could not be obtained by direct methods, intrinsic phasing, Patterson methods, or charge flipping. The indexing of the pattern showed it to have cubic metric symmetry (consistent with the optical behavior) with a=60.0168(13) Å. To corroborate the composition of the crystals, a portion was collected, dissolved in 550 μL of DMSO-d6, and analyzed by ¹H and ¹¹B NMR spectroscopy. The density of these crystals was measured by isopycnic flotation: bromoform and hexanes were mixed until a ratio was achieved where 1 mg of microcrystalline Na₂B₁₂I₁₂/γ-CD complex would remain suspended without sinking or rising. The entire microcrystalline sample achieved isopycnic flotation at the same bromoform:hexanes ratio. The mass of 250 μL of the solvent mixture was measured to determine its density.

Crystallography: Na₂B₁₂I_{12·}6DMF·H₂O. Crystals were obtained by allowing diethyl ether to diffuse into a DMF solution of the compound. A platy crystal was selected, mounted on a MiTeGen polyimide loop, and cooled to 100 K on a Rigaku Synergy-S X-ray diffractometer. Diffraction of Mo Kα radiation from a PhotonJet-S microfocus source was detected using a HyPix-6000HE hybrid photon counting detector. Screening, indexing, data collection, and data processing were performed with CrysAlis^{Pro}. The structure was solved using SHELXT and refined using SHELXL. All non-H atoms were refined anisotropically. Carbon-bound H atoms were placed at calculated positions and refined with a riding model and coupled isotropic displacement parameters (1.2×U_{eq} for DMF amide CHO groups and 1.5×U_{eq} for methyl groups). The calculated density of these crystals was obtained by dividing the mass of the unit cell contents by the unit cell volume.

Computational experiments. All calculations were performed in the gas phase using the PM6 semi-empirical method with Gaussian 16. The input geometry for the [(γ-CD)₂(B₁₂I₁₂)]²⁻ complex was generated from the coordinates of [(γ-CD)₂(B₁₂Br₁₂)]²⁻. The input geometry for the [(γ-CD)(B₁₂I₁₂)]²⁻ complex was obtained by removing one of the rings from the optimized geometry of the [(γ-CD)₂(B₁₂I₁₂)]²⁻. The input geometry for the [(HP-T-CD)(B₁₂I₁₂)]²⁻ complex was generated by adding 2-hydroxypropyl groups to the O6 positions of alternating glucose units. In all cases, geometry optimizations were performed under the constraint that C₂ symmetry be maintained.

### Results

Hemolytic Activity of Na₂B₁₂I₁₂ and Complexation with Cyclodextrin

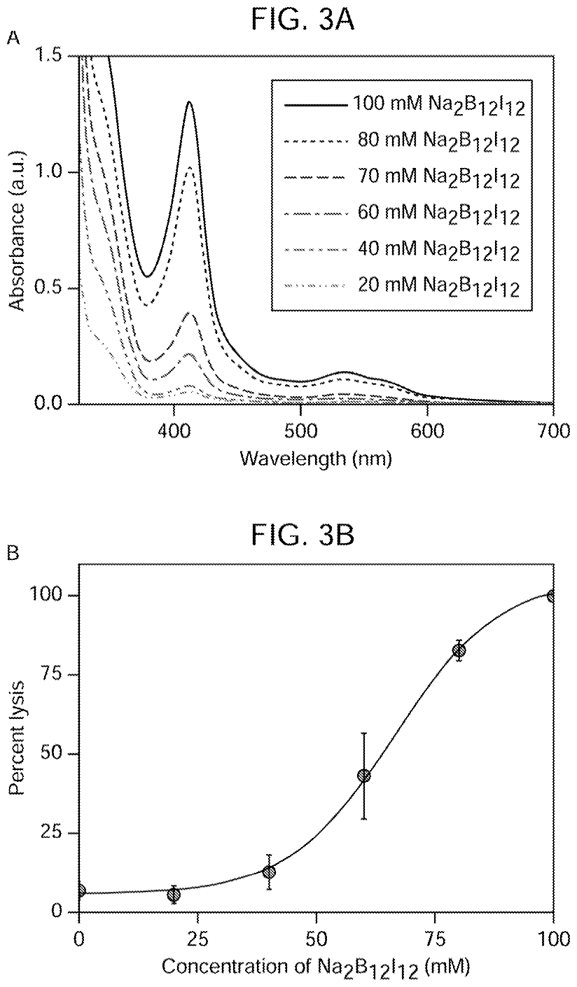

Exposure of human RBCs to 100 mM Na₂B₁₂I₁₂ resulted in rapid hemolysis, as determined by clarification of the suspension. The dose dependence of this hemolytic effect was explored by suspending RBCs in a PBS solution of Na₂B₁₂I₁₂ for 10 s, followed by rapid centrifugation to pellet the cells, and removal of the supernatant. The extent of hemolysis was determined by measuring the absorbance of the hemoglobin (Hb) in the supernatant (FIG. 3). As described herein, encapsulation within a supramolecular host would prevent damaging effects of the B₁₂I₁₂²⁻ anion where hemolytic activity stems from the chaotropic activity of the borate anions. The interaction of B₁₂I₁₂²⁻ with cyclodextrins (i.e., α-, β-, and γ-CD) was studied with isothermal calorimetry and interacts most strongly with γ-CD (FIG. 1; Kₐ=6.7×10⁴ L mol⁻¹).

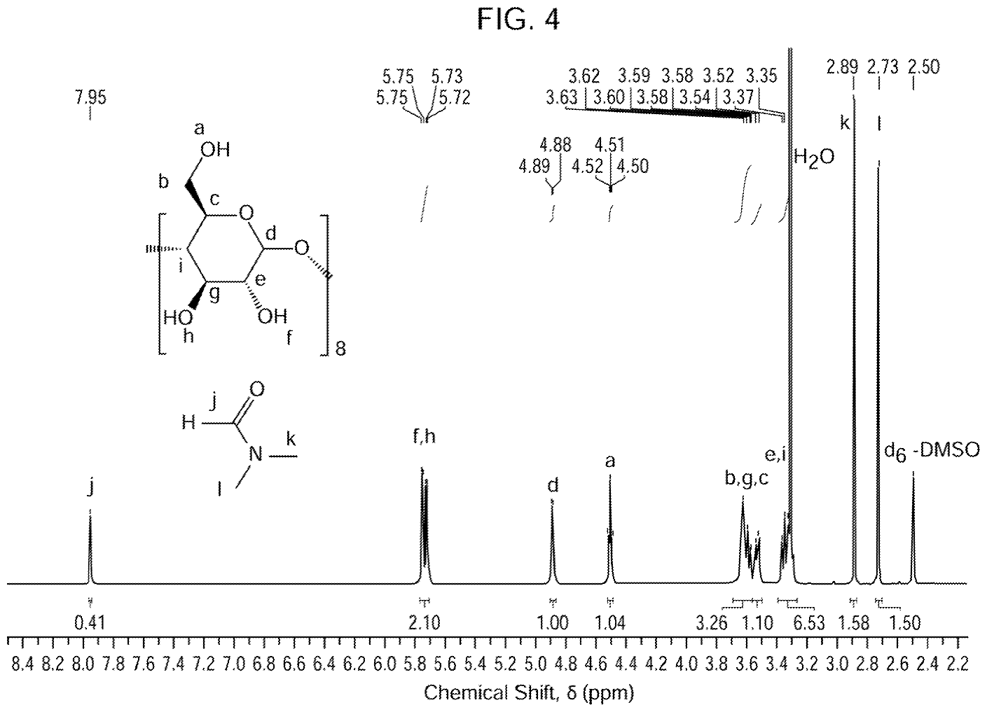

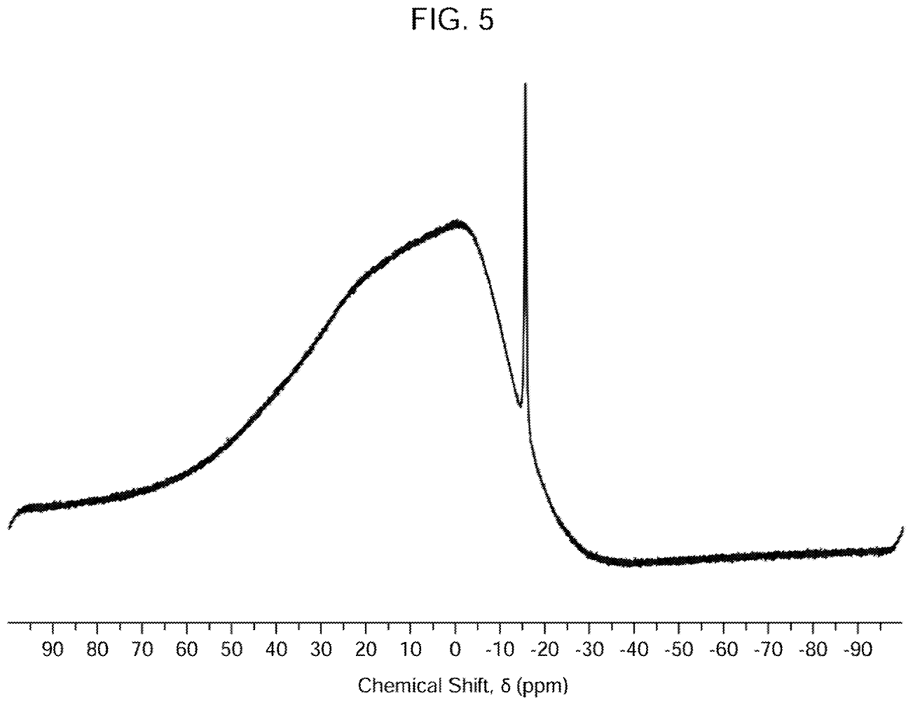

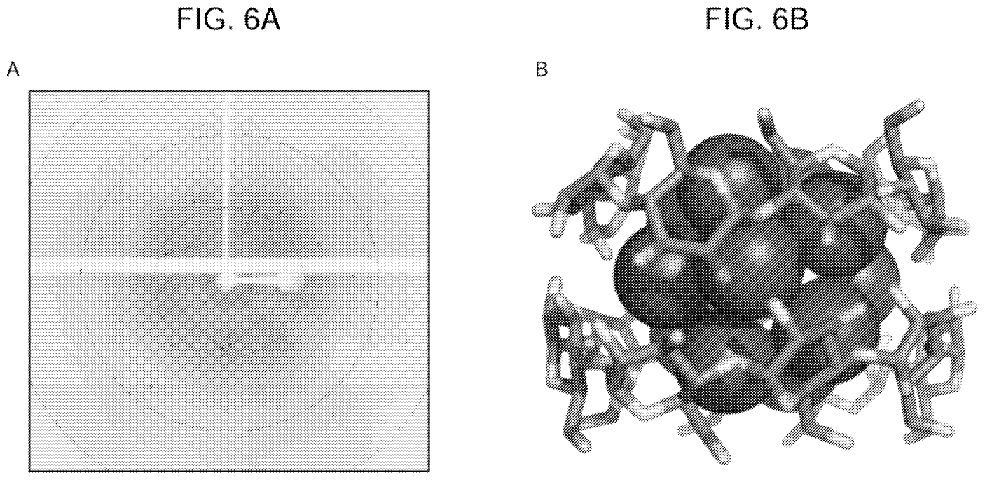

Single-crystal X-ray diffraction studies of B₁₂Br₁₂²⁻/γ-CD show the perbrominated cluster to form a 2:1 complex with the cyclic oligosaccharide in the solid state. Crystals were grown from a 2:1 mixture of γ-CD and Na₂B₁₂I₁₂ by allowing diethyl ether to diffuse into a DMF solution of the two species. ¹H and ¹¹B NMR spectra of solutions prepared from isolated crystals suggest that they contained both γ-CD and B₁₂I₁₂²⁻, along with 4 equiv of DMF with respect to γ-CD (FIGS. 4 and 5). To confirm that the sample was not a mixture of γ-CD crystals and Na₂B₁₂I₁₂ crystals, isopycnic flotation density measurements were performed. The crystals all exhibited the same isopycnic point in a mixture of bromoform and hexanes. The measured density (ρₑₓₚ=1.6 g mL⁻¹) was greater than the calculated density of γ-CD (1.41 g mL⁻¹ for the tetradecahydrate) and below the calculated density of Na₂B₁₂I₁₂·6DMF·H₂O) (2.19 g mL⁻¹, see ESI). This result is consistent with the present crystals containing both substances. The crystals did not diffract beyond 1.5 Å (FIG. 6A), and no solution could be obtained by direct methods, Patterson methods, intrinsic phasing, or charge flipping. FIG. 6C depicts thermal ellipsoid plot (50% probability level) of Na₂B₁₂I₁₂·6DMF·H₂O. Color code: B pink, I purple, Na teal, N blue, O red, C grey, H white spheres of arbitrary radius.

The diffraction pattern was indexed and exhibited cubic metric symmetry (a=60.0168(13) Å), which was consistent with the fact that the crystals were perpetually extinguished when viewed between crossed polarizers. The volume of the unit cell (216,181 Å³) is consistent with the composition (γ-CD)₂·Na₂B₁₂I₁₂·8DMF and Z=48, if the non-H atoms have the chemically reasonable average value of 19 Å^{3;18} the number of DMF molecules is consistent with the NMR data obtained from the crystals. The systematic absences and enantiomeric purity of the γ-CD narrow the possible space groups to F23 and F432. With Z=48, the complex would reside on a general position in the former and on a 2-fold axis in the latter. In summary, the aggregate crystallographic data suggest that the crystals may contain a 2:1 complex of the type observed for B₁₂Br₁₂². Table 1 summarizes the crystallographic details of the [(γ-CD)₂(B₁₂I₁₂)]²⁻ complex.

TABLE S1
Crystallographic details.

| Compound | Na₂[(γ-CD)₂(B₁₂I₁₂)] ^{a} | Na₂B₁₂I₁₂•6DMF•H₂O |
|---|---|---|
| Empirical formula | — | C₁₈H₄₄B₁₂I₁₂N₆Na₂O₇ |
| Formula weight | — | 2155.09 |
| Temperature (K) | 100(2) | 100(2) |
| Wavelength (Å) | 1.54184 | 0.71073 |
| Crystal system | Cubic | Orthorhombic |
| Space group | — | Pbcn |
| a (Å) | 60.0168(13) | 28.8693(6) |
| b (Å) | | 22.9629(4) |
| c (Å) | | 19.7457(3) |
| Volume (Å³) | 216181(8) | 13089.9(4) |
| Z | — | 8 |
| ρ_{calc} (Mg/m³) | — | 2.187 |
| Crystal size (mm³) | 0.18 × 0.18 × 0.14 | 0.22 × 0.19 × 0.04 |
| θ range (°) | 2.442 to 30.917 | 2.170 to 25.681 |
| Reflections | 27417 | 174695 |
| collected | | |
| Independent | 5247 | 12413 |
| reflections | | |
| Parameters | — | 627 |
| Completeness (%) | 99.8 | 99.9 |
| Rᵢₙₜ | 0.0661 | 0.0623 |
| R₁ (I > 2σ) | — | 0.0589 |
| R₁ (all data) | — | 0.0767 |
| wR₂ (I > 2σ) | — | 0.1497 |
| wR₂ (all data) | — | 0.1659 |
| Goodness of fit, S | — | 1.119 |

^{a} No solved or refined structure; data collection and unit cell parameters only. Compound name based on tentative assignment of identity.

A semi-empirical (PM6) geometry optimization confirms that such a host-guest complex is a minimum on the potential energy surface of this supramolecular system (FIG. 6B). The structure depicted in FIG. 6B is a theoretically optimized structure that, although consistent with the data collected from the crystals, was not obtained by refinement of a full crystal structure against the observed structure factors. Table 2 provides the Cartesian coordinates (A) of the optimized (PM6) structure of the [(γ-CD)₂(B₁₂I₁₂)]²⁻ complex. Table 3 provides Cartesian coordinates (Å) of the optimized (PM6) structure of the [(γ-CD)(B₁₂I₁₂)]²⁻ complex. Table 4 provides Cartesian coordinates (A) of the optimized (PM6) structure of the [(HP-γ-CD)(B₁₂I₁₂)]²⁻ complex.

TABLE 2
Cartesian coordinates (Å) of the optimized (PM6) structure
of the [(γ-CD)₂(B₁₂I₁₂)]²⁻ complex

| | | | | | | | |
|---|---|---|---|---|---|---|---|
| O | 5.044700 | 2.653600 | 3.033900 | O | 5.044700 | 2.653600 | 3.033900 |
| O | 6.778100 | 1.896100 | 1.200700 | O | 6.778100 | 1.896100 | 1.200700 |
| O | 6.759600 | −1.122400 | 1.262500 | O | 6.759600 | −1.122400 | 1.262500 |
| O | 4.494700 | −2.064600 | 2.721000 | O | 4.494700 | −2.064600 | 2.721000 |
| O | 5.575500 | 0.981200 | 4.509100 | O | 5.575500 | 0.981200 | 4.509100 |
| O | 4.534400 | −1.696700 | 5.826800 | O | 4.534400 | −1.696700 | 5.826800 |
| H | 4.331900 | −2.460500 | 5.219500 | H | 4.331900 | −2.460500 | 5.219500 |
| O | 5.004400 | −3.240500 | 0.497600 | O | 5.004400 | −3.240500 | 0.497600 |
| O | 3.051800 | −6.361200 | 5.507200 | O | 3.051800 | −6.361200 | 5.507200 |
| H | 2.459700 | −6.985900 | 5.002800 | H | 2.459700 | −6.985900 | 5.002800 |
| O | 4.450000 | −3.978500 | 4.001900 | O | 4.450000 | −3.978500 | 4.001900 |
| O | 4.695100 | −6.431200 | 0.978300 | O | 4.695100 | −6.431200 | 0.978300 |
| H | 4.902800 | −6.344800 | 0.006100 | H | 4.902800 | −6.344800 | 0.006100 |
| C | 6.115900 | 1.829100 | 3.492300 | C | 6.115900 | 1.829100 | 3.492300 |
| H | 6.898600 | 2.409300 | 4.027500 | H | 6.898600 | 2.409300 | 4.027500 |
| C | 6.686800 | 0.997600 | 2.308400 | C | 6.686800 | 0.997600 | 2.308400 |
| H | 7.732900 | 0.683800 | 2.554400 | H | 7.732900 | 0.683800 | 2.554400 |
| C | 5.845600 | −0.240200 | 1.944000 | C | 5.845600 | −0.240200 | 1.944000 |
| H | 4.992300 | 0.026300 | 1.250900 | H | 4.992300 | 0.026300 | 1.250900 |
| C | 5.341600 | −0.958000 | 3.208000 | C | 5.341600 | −0.958000 | 3.208000 |
| H | 6.176100 | −1.395100 | 3.804800 | H | 6.176100 | −1.395100 | 3.804800 |
| C | 4.541900 | 0.038900 | 4.057800 | C | 4.541900 | 0.038900 | 4.057800 |
| H | 3.784300 | 0.615900 | 3.450800 | H | 3.784300 | 0.615900 | 3.450800 |
| C | 3.912500 | −0.508200 | 5.343000 | C | 3.912500 | −0.508200 | 5.343000 |
| H | 4.057900 | 0.208300 | 6.181500 | H | 4.057900 | 0.208300 | 6.181500 |
| H | 2.829900 | −0.695300 | 5.182600 | H | 2.829900 | −0.695300 | 5.182600 |
| C | 5.116300 | −3.380400 | 2.923800 | C | 5.116300 | −3.380400 | 2.923800 |
| H | 6.150000 | −3.237500 | 3.322700 | H | 6.150000 | −3.237500 | 3.322700 |
| C | 5.090000 | −4.155100 | 1.587300 | C | 5.090000 | −4.155100 | 1.587300 |
| H | 6.098700 | −4.626700 | 1.430300 | H | 6.098700 | −4.626700 | 1.430300 |
| C | 4.012200 | −5.254100 | 1.446600 | C | 4.012200 | −5.254100 | 1.446600 |
| H | 3.217500 | −4.949900 | 0.704500 | H | 3.217500 | −4.949900 | 0.704500 |
| C | 3.353100 | −5.726600 | 2.755300 | C | 3.353100 | −5.726600 | 2.755300 |
| H | 3.923600 | −6.576400 | 3.199500 | H | 3.923600 | −6.576400 | 3.199500 |
| C | 2.739900 | −5.009000 | 5.187500 | C | 2.739900 | −5.009000 | 5.187500 |
| H | 3.337200 | −4.435800 | 5.934300 | H | 3.337200 | −4.435800 | 5.934300 |
| H | 1.663000 | −4.822300 | 5.351200 | H | 1.663000 | −4.822300 | 5.351200 |
| C | 3.146200 | −4.588900 | 3.763500 | C | 3.146200 | −4.588900 | 3.763500 |
| H | 2.444900 | −3.810700 | 3.353600 | H | 2.444900 | −3.810700 | 3.353600 |
| O | −2.012300 | 6.140600 | 2.356400 | O | −2.012300 | 6.140600 | 2.356400 |
| O | −1.170900 | 7.615800 | 0.176800 | O | −1.170900 | 7.615800 | 0.176800 |
| O | 1.713300 | 7.981200 | 1.010700 | O | 1.713300 | 7.981200 | 1.010700 |
| H | 1.436000 | 8.245600 | 0.071700 | H | 1.436000 | 8.245600 | 0.071700 |
| O | 2.175600 | 6.220900 | 3.143000 | O | 2.175600 | 6.220900 | 3.143000 |
| O | −1.138400 | 7.660800 | 3.851800 | O | −1.138400 | 7.660800 | 3.851800 |
| O | 4.261300 | 7.192900 | 1.887400 | O | 4.261300 | 7.192900 | 1.887400 |
| H | 3.284000 | 7.105100 | 1.622400 | H | 3.284000 | 7.105100 | 1.622400 |
| O | 4.450000 | 2.811600 | 6.310500 | O | 4.450000 | 2.811600 | 6.310500 |
| H | 4.989700 | 2.105800 | 5.849400 | H | 4.989700 | 2.105800 | 5.849400 |
| O | 3.324300 | 5.327800 | 4.899100 | O | 3.324300 | 5.327800 | 4.899100 |
| O | 6.414300 | 5.084100 | 2.303700 | O | 6.414300 | 5.084100 | 2.303700 |
| H | 6.533700 | 5.499800 | 1.393400 | H | 6.533700 | 5.499800 | 1.393400 |
| C | −1.705000 | 7.512700 | 2.545900 | C | −1.705000 | 7.512700 | 2.545900 |
| H | −2.603100 | 8.165300 | 2.579700 | H | −2.603100 | 8.165300 | 2.579700 |
| C | −0.671700 | 7.909700 | 1.467800 | C | −0.671700 | 7.909700 | 1.467800 |
| H | −0.550000 | 9.022900 | 1.453900 | H | −0.550000 | 9.022900 | 1.453900 |
| C | 0.695500 | 7.229900 | 1.681800 | C | 0.695500 | 7.229900 | 1.681800 |
| H | 0.703200 | 6.178400 | 1.289500 | H | 0.703200 | 6.178400 | 1.289500 |
| C | 1.170900 | 7.263500 | 3.154900 | C | 1.170900 | 7.263500 | 3.154900 |
| H | 1.630500 | 8.244700 | 3.405600 | H | 1.630500 | 8.244700 | 3.405600 |
| C | 0.053400 | 6.867900 | 4.136800 | C | 0.053400 | 6.867900 | 4.136800 |
| H | −0.195500 | 5.788400 | 4.010600 | H | −0.195500 | 5.788400 | 4.010600 |
| C | 3.331400 | 6.403900 | 3.984000 | C | 3.331400 | 6.403900 | 3.984000 |
| H | 3.248700 | 7.304100 | 4.636300 | H | 3.248700 | 7.304100 | 4.636300 |
| C | 4.555400 | 6.401600 | 3.041400 | C | 4.555400 | 6.401600 | 3.041400 |
| H | 5.417500 | 6.940800 | 3.508400 | H | 5.417500 | 6.940800 | 3.508400 |
| C | 5.015000 | 4.993800 | 2.598100 | C | 5.015000 | 4.993800 | 2.598100 |
| H | 4.445800 | 4.646800 | 1.696200 | H | 4.445800 | 4.646800 | 1.696200 |
| C | 4.910900 | 3.938500 | 3.716700 | C | 4.910900 | 3.938500 | 3.716700 |
| H | 5.709500 | 4.065600 | 4.480500 | H | 5.709500 | 4.065600 | 4.480500 |
| C | 3.268700 | 3.088800 | 5.565600 | C | 3.268700 | 3.088800 | 5.565600 |
| H | 2.627600 | 3.618900 | 6.301800 | H | 2.627600 | 3.618900 | 6.301800 |
| H | 2.785500 | 2.139200 | 5.259600 | H | 2.785500 | 2.139200 | 5.259600 |
| C | 3.503800 | 3.986900 | 4.340700 | C | 3.503800 | 3.986900 | 4.340700 |
| H | 2.714100 | 3.825700 | 3.557100 | H | 2.714100 | 3.825700 | 3.557100 |
| O | 2.012300 | −6.140600 | 2.356400 | O | 2.012300 | −6.140600 | 2.356400 |
| O | 1.170900 | −7.615800 | 0.176800 | O | 1.170900 | −7.615800 | 0.176800 |
| O | −1.713300 | −7.981200 | 1.010700 | O | −1.713300 | −7.981200 | 1.010700 |
| H | −1.436000 | −8.245600 | 0.071700 | H | −1.436000 | −8.245600 | 0.071700 |
| O | −2.175600 | −6.220900 | 3.143000 | O | −2.175600 | −6.220900 | 3.143000 |
| O | 1.138400 | −7.660800 | 3.851800 | O | 1.138400 | −7.660800 | 3.851800 |
| O | −1.614400 | −6.999300 | 6.050000 | O | −1.614400 | −6.999300 | 6.050000 |
| H | −1.823700 | −6.024700 | 5.992600 | H | −1.823700 | −6.024700 | 5.992600 |
| O | −4.261300 | −7.192900 | 1.887400 | O | −4.261300 | −7.192900 | 1.887400 |
| H | −3.284000 | −7.105100 | 1.622400 | H | −3.284000 | −7.105100 | 1.622400 |
| O | −4.450000 | −2.811600 | 6.310500 | O | −4.450000 | −2.811600 | 6.310500 |
| H | −4.989700 | −2.105800 | 5.849400 | H | −4.989700 | −2.105800 | 5.849400 |
| O | −3.324300 | −5.327800 | 4.899100 | O | −3.324300 | −5.327800 | 4.899100 |
| O | −6.414300 | −5.084100 | 2.303700 | O | −6.414300 | −5.084100 | 2.303700 |
| H | −6.533700 | −5.499800 | 1.393400 | H | −6.533700 | −5.499800 | 1.393400 |
| C | 1.705000 | −7.512700 | 2.545900 | C | 1.705000 | −7.512700 | 2.545900 |
| H | 2.603100 | −8.165300 | 2.579700 | H | 2.603100 | −8.165300 | 2.579700 |
| C | 0.671700 | −7.909700 | 1.467800 | C | 0.671700 | −7.909700 | 1.467800 |
| H | 0.550000 | −9.022900 | 1.453900 | H | 0.550000 | −9.022900 | 1.453900 |
| C | −0.695500 | −7.229900 | 1.681800 | C | −0.695500 | −7.229900 | 1.681800 |
| H | −0.703200 | −6.178400 | 1.289500 | H | −0.703200 | −6.178400 | 1.289500 |
| C | −1.170900 | −7.263500 | 3.154900 | C | −1.170900 | −7.263500 | 3.154900 |
| H | −1.630500 | −8.244700 | 3.405600 | H | −1.630500 | −8.244700 | 3.405600 |
| C | −0.053400 | −6.867900 | 4.136800 | C | −0.053400 | −6.867900 | 4.136800 |
| H | 0.195500 | −5.788400 | 4.010600 | H | 0.195500 | −5.788400 | 4.010600 |
| C | −0.291500 | −7.268300 | 5.608300 | C | −0.291500 | −7.268300 | 5.608300 |
| H | −0.194500 | −8.366400 | 5.737900 | H | −0.194500 | −8.366400 | 5.737900 |
| H | 0.436100 | −6.763200 | 6.272400 | H | 0.436100 | −6.763200 | 6.272400 |
| C | −3.331400 | −6.403900 | 3.984000 | C | −3.331400 | −6.403900 | 3.984000 |
| H | −3.248700 | −7.304100 | 4.636300 | H | −3.248700 | −7.304100 | 4.636300 |
| C | −4.555400 | −6.401600 | 3.041400 | C | −4.555400 | −6.401600 | 3.041400 |
| H | −5.417500 | −6.940800 | 3.508400 | H | −5.417500 | −6.940800 | 3.508400 |
| C | −5.015000 | −4.993800 | 2.598100 | C | −5.015000 | −4.993800 | 2.598100 |
| H | −4.445800 | −4.646800 | 1.696200 | H | −4.445800 | −4.646800 | 1.696200 |
| C | −4.910900 | −3.938500 | 3.716700 | C | −4.910900 | −3.938500 | 3.716700 |
| H | −5.709500 | −4.065600 | 4.480500 | H | −5.709500 | −4.065600 | 4.480500 |
| C | −3.268700 | −3.088800 | 5.565600 | C | −3.268700 | −3.088800 | 5.565600 |
| H | −2.627600 | −3.618900 | 6.301800 | H | −2.627600 | −3.618900 | 6.301800 |
| H | −2.785500 | −2.139200 | 5.259600 | H | −2.785500 | −2.139200 | 5.259600 |
| C | −3.503800 | −3.986900 | 4.340700 | C | −3.503800 | −3.986900 | 4.340700 |
| H | −2.714100 | −3.825700 | 3.557100 | H | −2.714100 | −3.825700 | 3.557100 |
| O | −5.044700 | −2.653600 | 3.033900 | O | −5.044700 | −2.653600 | 3.033900 |
| O | −6.778100 | −1.896100 | 1.200700 | O | −6.778100 | −1.896100 | 1.200700 |
| O | −6.759600 | 1.122400 | 1.262500 | O | −6.759600 | 1.122400 | 1.262500 |
| O | −4.494700 | 2.064600 | 2.721000 | O | −4.494700 | 2.064600 | 2.721000 |
| O | −5.575500 | −0.981200 | 4.509100 | O | −5.575500 | −0.981200 | 4.509100 |
| O | −4.534400 | 1.696700 | 5.826800 | O | −4.534400 | 1.696700 | 5.826800 |
| H | −4.331900 | 2.460500 | 5.219500 | H | −4.331900 | 2.460500 | 5.219500 |
| O | −5.004400 | 3.240500 | 0.497600 | O | −5.004400 | 3.240500 | 0.497600 |
| O | −3.051800 | 6.361200 | 5.507200 | O | −3.051800 | 6.361200 | 5.507200 |
| H | −2.459700 | 6.985900 | 5.002800 | H | −2.459700 | 6.985900 | 5.002800 |
| O | −4.450000 | 3.978500 | 4.001900 | O | −4.450000 | 3.978500 | 4.001900 |
| O | −4.695100 | 6.431200 | 0.978300 | O | −4.695100 | 6.431200 | 0.978300 |
| H | −4.902800 | 6.344800 | 0.006100 | H | −4.902800 | 6.344800 | 0.006100 |
| C | −6.115900 | −1.829100 | 3.492300 | C | −6.115900 | −1.829100 | 3.492300 |
| H | −6.898600 | −2.409300 | 4.027500 | H | −6.898600 | −2.409300 | 4.027500 |
| C | −6.686800 | −0.997600 | 2.308400 | C | −6.686800 | −0.997600 | 2.308400 |
| H | −7.732900 | −0.683800 | 2.554400 | H | −7.732900 | −0.683800 | 2.554400 |
| C | −5.845600 | 0.240200 | 1.944000 | C | −5.845600 | 0.240200 | 1.944000 |
| H | −4.992300 | −0.026300 | 1.250900 | H | −4.992300 | −0.026300 | 1.250900 |
| C | −5.341600 | 0.958000 | 3.208000 | C | −5.341600 | 0.958000 | 3.208000 |
| H | −6.176100 | 1.395100 | 3.804800 | H | −6.176100 | 1.395100 | 3.804800 |
| C | −4.541900 | −0.038900 | 4.057800 | C | −4.541900 | −0.038900 | 4.057800 |
| H | −3.784300 | −0.615900 | 3.450800 | H | −3.784300 | −0.615900 | 3.450800 |
| C | −3.912500 | 0.508200 | 5.343000 | C | −3.912500 | 0.508200 | 5.343000 |
| H | −4.057900 | −0.208300 | 6.181500 | H | −4.057900 | −0.208300 | 6.181500 |
| H | −2.829900 | 0.695300 | 5.182600 | H | −2.829900 | 0.695300 | 5.182600 |
| C | −5.116300 | 3.380400 | 2.923800 | C | −5.116300 | 3.380400 | 2.923800 |
| H | −6.150000 | 3.237500 | 3.322700 | H | −6.150000 | 3.237500 | 3.322700 |
| C | −5.090000 | 4.155100 | 1.587300 | C | −5.090000 | 4.155100 | 1.587300 |
| H | −6.098700 | 4.626700 | 1.430300 | H | −6.098700 | 4.626700 | 1.430300 |
| C | −4.012200 | 5.254100 | 1.446600 | C | −4.012200 | 5.254100 | 1.446600 |
| H | −3.217500 | 4.949900 | 0.704500 | H | −3.217500 | 4.949900 | 0.704500 |
| C | −3.353100 | 5.726600 | 2.755300 | C | −3.353100 | 5.726600 | 2.755300 |
| H | −3.923600 | 6.576400 | 3.199500 | H | −3.923600 | 6.576400 | 3.199500 |
| C | −2.739900 | 5.009000 | 5.187500 | C | −2.739900 | 5.009000 | 5.187500 |
| H | −3.337200 | 4.435800 | 5.934300 | H | −3.337200 | 4.435800 | 5.934300 |
| H | −1.663000 | 4.822300 | 5.351200 | H | −1.663000 | 4.822300 | 5.351200 |
| C | −3.146200 | 4.588900 | 3.763500 | C | −3.146200 | 4.588900 | 3.763500 |
| H | −2.444900 | 3.810700 | 3.353600 | H | −2.444900 | 3.810700 | 3.353600 |
| O | 5.100100 | 1.929900 | −1.894800 | O | 5.100100 | 1.929900 | −1.894800 |
| O | 7.168000 | 0.209500 | −0.972500 | O | 7.168000 | 0.209500 | −0.972500 |
| O | 6.570200 | −2.681400 | −1.595000 | O | 6.570200 | −2.681400 | −1.595000 |
| H | 5.665500 | −2.889500 | −1.200700 | H | 5.665500 | −2.889500 | −1.200700 |
| O | 4.388400 | −2.672600 | −2.997500 | O | 4.388400 | −2.672600 | −2.997500 |
| O | 4.982900 | −0.863500 | −4.347000 | O | 4.982900 | −0.863500 | −4.347000 |
| O | 5.700600 | 1.952500 | −5.308000 | O | 5.700600 | 1.952500 | −5.308000 |
| H | 6.052000 | 2.524800 | −4.569500 | H | 6.052000 | 2.524800 | −4.569500 |
| O | 5.449000 | −5.647200 | −1.906100 | O | 5.449000 | −5.647200 | −1.906100 |
| H | 6.222300 | −5.034100 | −1.809000 | H | 6.222300 | −5.034100 | −1.809000 |
| O | 2.220500 | −5.472200 | −1.011800 | O | 2.220500 | −5.472200 | −1.011800 |
| O | 2.876600 | −5.647400 | −4.494500 | O | 2.876600 | −5.647400 | −4.494500 |
| O | 4.694600 | −4.301500 | −6.249900 | O | 4.694600 | −4.301500 | −6.249900 |
| H | 4.612800 | −5.282000 | −6.282600 | H | 4.612800 | −5.282000 | −6.282600 |
| C | 5.673000 | 0.822300 | −2.684500 | C | 5.673000 | 0.822300 | −2.684500 |
| H | 6.594400 | 1.180600 | −3.205700 | H | 6.594400 | 1.180600 | −3.205700 |
| C | 6.030800 | −0.298700 | −1.699700 | C | 6.030800 | −0.298700 | −1.699700 |
| H | 5.194100 | −0.505100 | −0.981300 | H | 5.194100 | −0.505100 | −0.981300 |
| C | 6.469200 | −1.562100 | −2.467700 | C | 6.469200 | −1.562100 | −2.467700 |
| H | 7.514600 | −1.443800 | −2.846500 | H | 7.514600 | −1.443800 | −2.846500 |
| C | 5.508700 | −1.916400 | −3.626000 | C | 5.508700 | −1.916400 | −3.626000 |
| H | 5.957500 | −2.572900 | −4.410300 | H | 5.957500 | −2.572900 | −4.410300 |
| C | 4.607000 | 0.399000 | −3.699100 | C | 4.607000 | 0.399000 | −3.699100 |
| H | 3.597900 | 0.242600 | −3.206600 | H | 3.597900 | 0.242600 | −3.206600 |
| C | 4.474000 | 1.332400 | −4.909700 | C | 4.474000 | 1.332400 | −4.909700 |
| H | 4.184100 | 0.750800 | −5.810800 | H | 4.184100 | 0.750800 | −5.810800 |
| H | 3.718400 | 2.115700 | −4.701400 | H | 3.718400 | 2.115700 | −4.701400 |
| C | 4.348300 | −4.085000 | −3.418300 | C | 4.348300 | −4.085000 | −3.418300 |
| H | 5.297200 | −4.332000 | −3.955600 | H | 5.297200 | −4.332000 | −3.955600 |
| C | 4.243400 | −4.879800 | −2.111700 | C | 4.243400 | −4.879800 | −2.111700 |
| H | 4.145500 | −4.161500 | −1.239700 | H | 4.145500 | −4.161500 | −1.239700 |
| C | 3.120200 | −5.931000 | −2.011000 | C | 3.120200 | −5.931000 | −2.011000 |
| H | 3.539300 | −6.879600 | −1.580700 | H | 3.539300 | −6.879600 | −1.580700 |
| C | 2.352100 | −6.255200 | −3.316600 | C | 2.352100 | −6.255200 | −3.316600 |
| H | 2.322200 | −7.336400 | −3.572400 | H | 2.322200 | −7.336400 | −3.572400 |
| C | 3.144200 | −4.223500 | −4.359500 | C | 3.144200 | −4.223500 | −4.359500 |
| H | 2.224300 | −3.718200 | −3.951100 | H | 2.224300 | −3.718200 | −3.951100 |
| C | 3.462200 | −3.740400 | −5.778000 | C | 3.462200 | −3.740400 | −5.778000 |
| H | 2.648800 | −3.990500 | −6.484400 | H | 2.648800 | −3.990500 | −6.484400 |
| H | 3.653600 | −2.643600 | −5.785300 | H | 3.653600 | −2.643600 | −5.785300 |
| O | −1.053400 | 5.683200 | −3.135400 | O | −1.053400 | 5.683200 | −3.135400 |
| O | −0.406600 | 8.458900 | −2.364000 | O | −0.406600 | 8.458900 | −2.364000 |
| H | −0.784400 | 8.552500 | −1.446300 | H | −0.784400 | 8.552500 | −1.446300 |
| O | 2.481200 | 8.310600 | −1.345200 | O | 2.481200 | 8.310600 | −1.345200 |
| H | 2.960500 | 7.522900 | −0.941700 | H | 2.960500 | 7.522900 | −0.941700 |
| O | 3.307000 | 6.102800 | −2.264400 | O | 3.307000 | 6.102800 | −2.264400 |
| O | 2.320100 | 6.229000 | −4.358000 | O | 2.320100 | 6.229000 | −4.358000 |
| O | −0.249900 | 5.390700 | −6.100300 | O | −0.249900 | 5.390700 | −6.100300 |
| H | −1.107800 | 5.398300 | −5.594200 | H | −1.107800 | 5.398300 | −5.594200 |
| O | 5.672300 | 6.177000 | −0.182800 | O | 5.672300 | 6.177000 | −0.182800 |
| H | 5.105700 | 6.785400 | 0.393700 | H | 5.105700 | 6.785400 | 0.393700 |
| O | 4.796000 | 3.034400 | 0.214500 | O | 4.796000 | 3.034400 | 0.214500 |
| O | 6.010200 | 3.744900 | −3.040200 | O | 6.010200 | 3.744900 | −3.040200 |
| O | 5.857000 | 6.087400 | −4.806500 | O | 5.857000 | 6.087400 | −4.806500 |
| H | 6.747700 | 5.818700 | −4.487900 | H | 6.747700 | 5.818700 | −4.487900 |
| C | 0.052400 | 6.499400 | −3.594600 | C | 0.052400 | 6.499400 | −3.594600 |
| H | −0.262900 | 7.148900 | −4.445200 | H | −0.262900 | 7.148900 | −4.445200 |
| C | 0.509300 | 7.351700 | −2.396100 | C | 0.509300 | 7.351700 | −2.396100 |
| H | 0.444400 | 6.772500 | −1.445300 | H | 0.444400 | 6.772500 | −1.445300 |
| C | 1.916400 | 7.949500 | −2.611100 | C | 1.916400 | 7.949500 | −2.611100 |
| H | 1.834000 | 8.925500 | −3.148300 | H | 1.834000 | 8.925500 | −3.148300 |
| C | 2.904400 | 6.988200 | −3.320700 | C | 2.904400 | 6.988200 | −3.320700 |
| H | 3.767700 | 7.481800 | −3.818400 | H | 3.767700 | 7.481800 | −3.818400 |
| C | 1.116200 | 5.478700 | −4.025700 | C | 1.116200 | 5.478700 | −4.025700 |
| H | 1.362000 | 4.762000 | −3.198100 | H | 1.362000 | 4.762000 | −3.198100 |
| C | 0.769400 | 4.746000 | −5.333800 | C | 0.769400 | 4.746000 | −5.333800 |
| H | 1.639000 | 4.748400 | −6.022700 | H | 1.639000 | 4.748400 | −6.022700 |
| H | 0.447800 | 3.704500 | −5.117400 | H | 0.447800 | 3.704500 | −5.117400 |
| C | 4.679300 | 5.617700 | −2.281700 | C | 4.679300 | 5.617700 | −2.281700 |
| H | 5.367800 | 6.460000 | −2.529600 | H | 5.367800 | 6.460000 | −2.529600 |
| C | 4.870200 | 5.163600 | −0.823000 | C | 4.870200 | 5.163600 | −0.823000 |
| H | 3.864900 | 5.070100 | −0.324800 | H | 3.864900 | 5.070100 | −0.324800 |
| C | 5.668300 | 3.867900 | −0.581200 | C | 5.668300 | 3.867900 | −0.581200 |
| H | 6.587200 | 4.107500 | 0.016500 | H | 6.587200 | 4.107500 | 0.016500 |
| C | 6.049500 | 3.028300 | −1.811400 | C | 6.049500 | 3.028300 | −1.811400 |
| H | 7.098400 | 2.654000 | −1.782000 | H | 7.098400 | 2.654000 | −1.782000 |
| C | 4.778000 | 4.474800 | −3.303400 | C | 4.778000 | 4.474800 | −3.303400 |
| H | 3.914000 | 3.769600 | −3.197800 | H | 3.914000 | 3.769600 | −3.197800 |
| C | 4.971800 | 4.961700 | −4.746500 | C | 4.971800 | 4.961700 | −4.746500 |
| H | 5.365200 | 4.148200 | −5.392900 | H | 5.365200 | 4.148200 | −5.392900 |
| H | 4.023000 | 5.354100 | −5.175300 | H | 4.023000 | 5.354100 | −5.175300 |
| O | 1.053400 | −5.683200 | −3.135400 | O | 1.053400 | −5.683200 | −3.135400 |
| O | 0.406600 | −8.458900 | −2.364000 | O | 0.406600 | −8.458900 | −2.364000 |
| H | 0.784400 | −8.552500 | −1.446300 | H | 0.784400 | −8.552500 | −1.446300 |
| O | −2.481200 | −8.310600 | −1.345200 | O | −2.481200 | −8.310600 | −1.345200 |
| H | −2.960500 | −7.522900 | −0.941700 | H | −2.960500 | −7.522900 | −0.941700 |
| O | −3.307000 | −6.102800 | −2.264400 | O | −3.307000 | −6.102800 | −2.264400 |
| O | −2.320100 | −6.229000 | −4.358000 | O | −2.320100 | −6.229000 | −4.358000 |
| O | 0.249900 | −5.390700 | −6.100300 | O | 0.249900 | −5.390700 | −6.100300 |
| H | 1.107800 | −5.398300 | −5.594200 | H | 1.107800 | −5.398300 | −5.594200 |
| O | −5.672300 | −6.177000 | −0.182800 | O | −5.672300 | −6.177000 | −0.182800 |
| H | −5.105700 | −6.785400 | 0.393700 | H | −5.105700 | −6.785400 | 0.393700 |
| O | −4.796000 | −3.034400 | 0.214500 | O | −4.796000 | −3.034400 | 0.214500 |
| O | −6.010200 | −3.744900 | −3.040200 | O | −6.010200 | −3.744900 | −3.040200 |
| O | −5.857000 | −6.087400 | −4.806500 | O | −5.857000 | −6.087400 | −4.806500 |
| H | −6.747700 | −5.818700 | −4.487900 | H | −6.747700 | −5.818700 | −4.487900 |
| C | −0.052400 | −6.499400 | −3.594600 | C | −0.052400 | −6.499400 | −3.594600 |
| H | 0.262900 | −7.148900 | −4.445200 | H | 0.262900 | −7.148900 | −4.445200 |
| C | −0.509300 | −7.351700 | −2.396100 | C | −0.509300 | −7.351700 | −2.396100 |
| H | −0.444400 | −6.772500 | −1.445300 | H | −0.444400 | −6.772500 | −1.445300 |
| C | −1.916400 | −7.949500 | −2.611100 | C | −1.916400 | −7.949500 | −2.611100 |
| H | −1.834000 | −8.925500 | −3.148300 | H | −1.834000 | −8.925500 | −3.148300 |
| C | −2.904400 | −6.988200 | −3.320700 | C | −2.904400 | −6.988200 | −3.320700 |
| H | −3.767700 | −7.481800 | −3.818400 | H | −3.767700 | −7.481800 | −3.818400 |
| C | −1.116200 | −5.478700 | −4.025700 | C | −1.116200 | −5.478700 | −4.025700 |
| H | −1.362000 | −4.762000 | −3.198100 | H | −1.362000 | −4.762000 | −3.198100 |
| C | −0.769400 | −4.746000 | −5.333800 | C | −0.769400 | −4.746000 | −5.333800 |
| H | −1.639000 | −4.748400 | −6.022700 | H | −1.639000 | −4.748400 | −6.022700 |
| H | −0.447800 | −3.704500 | −5.117400 | H | −0.447800 | −3.704500 | −5.117400 |
| C | −4.679300 | −5.617700 | −2.281700 | C | −4.679300 | −5.617700 | −2.281700 |
| H | −5.367800 | −6.460000 | −2.529600 | H | −5.367800 | −6.460000 | −2.529600 |
| C | −4.870200 | −5.163600 | −0.823000 | C | −4.870200 | −5.163600 | −0.823000 |
| H | −3.864900 | −5.070100 | −0.324800 | H | −3.864900 | −5.070100 | −0.324800 |
| C | −5.668300 | −3.867900 | −0.581200 | C | −5.668300 | −3.867900 | −0.581200 |
| H | −6.587200 | −4.107500 | 0.016500 | H | −6.587200 | −4.107500 | 0.016500 |
| C | −6.049500 | −3.028300 | −1.811400 | C | −6.049500 | −3.028300 | −1.811400 |
| H | −7.098400 | −2.654000 | −1.782000 | H | −7.098400 | −2.654000 | −1.782000 |
| C | −4.778000 | −4.474800 | −3.303400 | C | −4.778000 | −4.474800 | −3.303400 |
| H | −3.914000 | −3.769600 | −3.197800 | H | −3.914000 | −3.769600 | −3.197800 |
| C | −4.971800 | −4.961700 | −4.746500 | C | −4.971800 | −4.961700 | −4.746500 |
| H | −5.365200 | −4.148200 | −5.392900 | H | −5.365200 | −4.148200 | −5.392900 |
| H | −4.023000 | −5.354100 | −5.175300 | H | −4.023000 | −5.354100 | −5.175300 |
| O | −5.100100 | −1.929900 | −1.894800 | O | −5.100100 | −1.929900 | −1.894800 |
| O | −7.168000 | −0.209500 | −0.972500 | O | −7.168000 | −0.209500 | −0.972500 |
| O | −6.570200 | 2.681400 | −1.595000 | O | −6.570200 | 2.681400 | −1.595000 |
| H | −5.665500 | 2.889500 | −1.200700 | H | −5.665500 | 2.889500 | −1.200700 |
| O | −4.388400 | 2.672600 | −2.997500 | O | −4.388400 | 2.672600 | −2.997500 |
| O | −4.982900 | 0.863500 | −4.347000 | O | −4.982900 | 0.863500 | −4.347000 |
| O | −5.700600 | −1.952500 | −5.308000 | O | −5.700600 | −1.952500 | −5.308000 |
| H | −6.052000 | −2.524800 | −4.569500 | H | −6.052000 | −2.524800 | −4.569500 |
| O | −5.449000 | 5.647200 | −1.906100 | O | −5.449000 | 5.647200 | −1.906100 |
| H | −6.222300 | 5.034100 | −1.809000 | H | −6.222300 | 5.034100 | −1.809000 |
| O | −2.220500 | 5.472200 | −1.011800 | O | −2.220500 | 5.472200 | −1.011800 |
| O | −2.876600 | 5.647400 | −4.494500 | O | −2.876600 | 5.647400 | −4.494500 |
| O | −4.694600 | 4.301500 | −6.249900 | O | −4.694600 | 4.301500 | −6.249900 |
| H | −4.612800 | 5.282000 | −6.282600 | H | −4.612800 | 5.282000 | −6.282600 |
| C | −5.673000 | −0.822300 | −2.684500 | C | −5.673000 | −0.822300 | −2.684500 |
| H | −6.594400 | −1.180600 | −3.205700 | H | −6.594400 | −1.180600 | −3.205700 |
| C | −6.030800 | 0.298700 | −1.699700 | C | −6.030800 | 0.298700 | −1.699700 |
| H | −5.194100 | 0.505100 | −0.981300 | H | −5.194100 | 0.505100 | −0.981300 |
| C | −6.469200 | 1.562100 | −2.467700 | C | −6.469200 | 1.562100 | −2.467700 |
| H | −7.514600 | 1.443800 | −2.846500 | H | −7.514600 | 1.443800 | −2.846500 |
| C | −5.508700 | 1.916400 | −3.626000 | C | −5.508700 | 1.916400 | −3.626000 |
| H | −5.957500 | 2.572900 | −4.410300 | H | −5.957500 | 2.572900 | −4.410300 |
| C | −4.607000 | −0.399000 | −3.699100 | C | −4.607000 | −0.399000 | −3.699100 |
| H | −3.597900 | −0.242600 | −3.206600 | H | −3.597900 | −0.242600 | −3.206600 |
| C | −4.474000 | −1.332400 | −4.909700 | C | −4.474000 | −1.332400 | −4.909700 |
| H | −4.184100 | −0.750800 | −5.810800 | H | −4.184100 | −0.750800 | −5.810800 |
| H | −3.718400 | −2.115700 | −4.701400 | H | −3.718400 | −2.115700 | −4.701400 |
| C | −4.348300 | 4.085000 | −3.418300 | C | −4.348300 | 4.085000 | −3.418300 |
| H | −5.297200 | 4.332000 | −3.955600 | H | −5.297200 | 4.332000 | −3.955600 |
| C | −4.243400 | 4.879800 | −2.111700 | C | −4.243400 | 4.879800 | −2.111700 |
| H | −4.145500 | 4.161500 | −1.239700 | H | −4.145500 | 4.161500 | −1.239700 |
| C | −3.120200 | 5.931000 | −2.011000 | C | −3.120200 | 5.931000 | −2.011000 |
| H | −3.539300 | 6.879600 | −1.580700 | H | −3.539300 | 6.879600 | −1.580700 |
| C | −2.352100 | 6.255200 | −3.316600 | C | −2.352100 | 6.255200 | −3.316600 |
| H | −2.322200 | 7.336400 | −3.572400 | H | −2.322200 | 7.336400 | −3.572400 |
| C | −3.144200 | 4.223500 | −4.359500 | C | −3.144200 | 4.223500 | −4.359500 |
| H | −2.224300 | 3.718200 | −3.951100 | H | −2.224300 | 3.718200 | −3.951100 |
| C | −3.462200 | 3.740400 | −5.778000 | C | −3.462200 | 3.740400 | −5.778000 |
| H | −2.648800 | 3.990500 | −6.484400 | H | −2.648800 | 3.990500 | −6.484400 |
| H | −3.653600 | 2.643600 | −5.785300 | H | −3.653600 | 2.643600 | −5.785300 |
| I | 2.693700 | −1.840100 | −2.037600 | I | 2.693700 | −1.840100 | −2.037600 |
| I | 3.411200 | 1.634800 | −0.417400 | I | 3.411200 | 1.634800 | −0.417400 |
| I | 1.064100 | 1.641600 | 2.988400 | I | 1.064100 | 1.641600 | 2.988400 |
| B | 1.170900 | −0.796000 | −1.055900 | B | 1.170900 | −0.796000 | −1.055900 |
| B | 1.559500 | 0.680600 | −0.210300 | B | 1.559500 | 0.680600 | −0.210300 |
| B | 0.504500 | 0.714500 | 1.186900 | B | 0.504500 | 0.714500 | 1.186900 |
| I | 1.007000 | 1.622600 | −3.403700 | I | 1.007000 | 1.622600 | −3.403700 |
| I | 0.210100 | 3.732600 | −0.114400 | I | 0.210100 | 3.732600 | −0.114400 |
| I | −2.611700 | 1.603800 | 1.894500 | I | −2.611700 | 1.603800 | 1.894500 |
| B | 0.492400 | 0.706400 | −1.584900 | B | 0.492400 | 0.706400 | −1.584900 |
| B | 0.089700 | 1.630500 | −0.199500 | B | 0.089700 | 1.630500 | −0.199500 |
| B | −1.150300 | 0.777700 | 0.661500 | B | −1.150300 | 0.777700 | 0.661500 |
| I | −2.693700 | 1.840100 | −2.037600 | I | −2.693700 | 1.840100 | −2.037600 |
| I | −3.411200 | −1.634800 | −0.417400 | I | −3.411200 | −1.634800 | −0.417400 |
| I | −1.064100 | −1.641600 | 2.988400 | I | −1.064100 | −1.641600 | 2.988400 |
| B | −1.170900 | 0.796000 | −1.055900 | B | −1.170900 | 0.796000 | −1.055900 |
| B | −1.559500 | −0.680600 | −0.210300 | B | −1.559500 | −0.680600 | −0.210300 |
| B | −0.504500 | −0.714500 | 1.186900 | B | −0.504500 | −0.714500 | 1.186900 |
| I | −1.007000 | −1.622600 | −3.403700 | I | −1.007000 | −1.622600 | −3.403700 |
| I | −0.210100 | −3.732600 | −0.114400 | I | −0.210100 | −3.732600 | −0.114400 |
| I | 2.611700 | −1.603800 | 1.894500 | I | 2.611700 | −1.603800 | 1.894500 |
| B | −0.492400 | −0.706400 | −1.584900 | B | −0.492400 | −0.706400 | −1.584900 |
| B | −0.089700 | −1.630500 | −0.199500 | B | −0.089700 | −1.630500 | −0.199500 |
| B | 1.150300 | −0.777700 | 0.661500 | B | 1.150300 | −0.777700 | 0.661500 |
| C | 0.291500 | 7.268300 | 5.608300 | C | 0.291500 | 7.268300 | 5.608300 |
| H | −0.436100 | 6.763200 | 6.272400 | H | −0.436100 | 6.763200 | 6.272400 |
| H | 0.194500 | 8.366400 | 5.737900 | H | 0.194500 | 8.366400 | 5.737900 |
| O | 1.614400 | 6.999300 | 6.050000 | O | 1.614400 | 6.999300 | 6.050000 |
| H | 1.823700 | 6.024700 | 5.992600 | H | 1.823700 | 6.024700 | 5.992600 |
| H | 6.741100 | 1.372000 | 0.314000 | H | 6.741100 | 1.372000 | 0.314000 |
| H | 6.233500 | −1.903100 | 0.872000 | H | 6.233500 | −1.903100 | 0.872000 |
| H | 4.097000 | −2.720400 | 0.501800 | H | 4.097000 | −2.720400 | 0.501800 |
| H | −1.504700 | 6.649300 | 0.104700 | H | −1.504700 | 6.649300 | 0.104700 |
| H | 1.504700 | −6.649300 | 0.104700 | H | 1.504700 | −6.649300 | 0.104700 |
| H | −6.741100 | −1.372000 | 0.314000 | H | −6.741100 | −1.372000 | 0.314000 |
| H | −6.233500 | 1.903100 | 0.872000 | H | −6.233500 | 1.903100 | 0.872000 |
| H | −4.097000 | 2.720400 | 0.501800 | H | −4.097000 | 2.720400 | 0.501800 |
| H | 7.427700 | −0.463300 | −0.253100 | H | 7.427700 | −0.463300 | −0.253100 |
| H | 1.619300 | −4.695700 | −1.356500 | H | 1.619300 | −4.695700 | −1.356500 |
| H | 5.272800 | 2.767200 | 1.128500 | H | 5.272800 | 2.767200 | 1.128500 |
| H | −5.272800 | −2.767200 | 1.128500 | H | −5.272800 | −2.767200 | 1.128500 |
| H | −7.427700 | 0.463300 | −0.253100 | H | −7.427700 | 0.463300 | −0.253100 |
| H | −1.619300 | 4.695700 | −1.356500 | H | −1.619300 | 4.695700 | −1.356500 |

TABLE 3
Cartesian coordinates (Å) of the optimized (PM6) structure
of the [(γ-CD)(B₁₂I₁₂)]²⁻ complex.

| | | | | | | | |
|---|---|---|---|---|---|---|---|
| O | −5.470000 | −2.859300 | −1.354600 | O | −5.470000 | −2.859300 | −1.354600 |
| O | −7.501800 | −2.040200 | 0.593300 | O | −7.501800 | −2.040200 | 0.593300 |
| O | −8.034700 | 0.754500 | 0.374000 | O | −8.034700 | 0.754500 | 0.374000 |
| H | −8.323000 | 0.226000 | 1.164400 | H | −8.323000 | 0.226000 | 1.164400 |
| O | −6.183700 | 1.237600 | −1.834400 | O | −6.183700 | 1.237600 | −1.834400 |
| O | −7.340800 | −0.221500 | −3.074900 | O | −7.340800 | −0.221500 | −3.074900 |
| O | −6.577300 | −3.347100 | −4.157200 | O | −6.577300 | −3.347100 | −4.157200 |
| H | −5.845900 | −3.800600 | −3.646300 | H | −5.845900 | −3.800600 | −3.646300 |
| O | −5.164800 | 0.944600 | 0.840900 | O | −5.164800 | 0.944600 | 0.840900 |
| H | −6.151700 | 0.836400 | 1.035600 | H | −6.151700 | 0.836400 | 1.035600 |
| O | −3.423100 | 3.217400 | 1.568000 | O | −3.423100 | 3.217400 | 1.568000 |
| O | −5.364100 | 4.722100 | −1.038800 | O | −5.364100 | 4.722100 | −1.038800 |
| O | −7.946100 | 4.282400 | −2.198000 | O | −7.946100 | 4.282400 | −2.198000 |
| H | −7.796800 | 4.894700 | −1.441700 | H | −7.796800 | 4.894700 | −1.441700 |
| C | −6.663800 | −2.111800 | −1.691500 | C | −6.663800 | −2.111800 | −1.691500 |
| H | −7.513700 | −2.790800 | −1.917800 | H | −7.513700 | −2.790800 | −1.917800 |
| C | −6.920300 | −1.244100 | −0.441500 | C | −6.920300 | −1.244100 | −0.441500 |
| H | −5.947300 | −0.807600 | −0.079300 | H | −5.947300 | −0.807600 | −0.079300 |
| C | −7.948400 | −0.144600 | −0.747000 | C | −7.948400 | −0.144600 | −0.747000 |
| H | −8.960300 | −0.576000 | −0.921400 | H | −8.960300 | −0.576000 | −0.921400 |
| C | −7.495700 | 0.677300 | −1.985200 | C | −7.495700 | 0.677300 | −1.985200 |
| H | −8.227500 | 1.436400 | −2.324800 | H | −8.227500 | 1.436400 | −2.324800 |
| C | −6.291200 | −1.230900 | −2.900400 | C | −6.291200 | −1.230900 | −2.900400 |
| H | −5.325000 | −0.698800 | −2.715900 | H | −5.325000 | −0.698800 | −2.715900 |
| C | −6.313900 | −1.949100 | −4.261100 | C | −6.313900 | −1.949100 | −4.261100 |
| H | −7.158600 | −1.574900 | −4.879200 | H | −7.158600 | −1.574900 | −4.879200 |
| H | −5.362500 | −1.796000 | −4.801400 | H | −5.362500 | −1.796000 | −4.801400 |
| C | −6.077500 | 2.392800 | −0.977900 | C | −6.077500 | 2.392800 | −0.977900 |
| H | −7.045000 | 2.604200 | −0.473200 | H | −7.045000 | 2.604200 | −0.473200 |
| C | −4.929800 | 2.096600 | −0.001600 | C | −4.929800 | 2.096600 | −0.001600 |
| C | −4.689300 | 3.327700 | 0.897300 | C | −4.689300 | 3.327700 | 0.897300 |
| H | −5.497700 | 3.479500 | 1.636200 | H | −5.497700 | 3.479500 | 1.636200 |
| C | −4.433500 | 4.569000 | 0.021300 | C | −4.433500 | 4.569000 | 0.021300 |
| H | −4.477500 | 5.552400 | 0.542200 | H | −4.477500 | 5.552400 | 0.542200 |
| C | −5.616800 | 3.561200 | −1.879900 | C | −5.616800 | 3.561200 | −1.879900 |
| H | −4.655800 | 3.303500 | −2.413300 | H | −4.655800 | 3.303500 | −2.413300 |
| C | −6.691700 | 4.040900 | −2.855400 | C | −6.691700 | 4.040900 | −2.855400 |
| H | −6.377200 | 4.969800 | −3.365300 | H | −6.377200 | 4.969800 | −3.365300 |
| H | −6.933000 | 3.254400 | −3.597200 | H | −6.933000 | 3.254400 | −3.597200 |
| O | 3.057600 | −4.028300 | −1.602200 | O | 3.057600 | −4.028300 | −1.602200 |
| O | 2.715100 | −6.505500 | −0.039000 | O | 2.715100 | −6.505500 | −0.039000 |
| H | 3.376200 | −6.003000 | 0.535600 | H | 3.376200 | −6.003000 | 0.535600 |
| O | −0.264400 | −6.417500 | 0.850100 | O | −0.264400 | −6.417500 | 0.850100 |
| H | −0.370300 | −5.460900 | 1.162300 | H | −0.370300 | −5.460900 | 1.162300 |
| O | −1.582600 | −5.113900 | −0.906200 | O | −1.582600 | −5.113900 | −0.906200 |
| O | −0.150000 | −5.545700 | −2.691100 | O | −0.150000 | −5.545700 | −2.691100 |
| O | 2.450400 | −4.604300 | −4.526300 | O | 2.450400 | −4.604300 | −4.526300 |
| H | 3.168700 | −4.160800 | −3.997500 | H | 3.168700 | −4.160800 | −3.997500 |
| O | −3.873600 | −6.505800 | 0.919000 | O | −3.873600 | −6.505800 | 0.919000 |
| H | −3.032800 | −6.797400 | 1.348200 | H | −3.032800 | −6.797400 | 1.348200 |
| O | −5.095200 | −3.404600 | 1.046700 | O | −5.095200 | −3.404600 | 1.046700 |
| O | −4.989700 | −4.883900 | −2.232700 | O | −4.989700 | −4.883900 | −2.232700 |
| O | −3.452400 | −6.605900 | −3.739000 | O | −3.452400 | −6.605900 | −3.739000 |
| H | −4.412600 | −6.725200 | −3.554400 | H | −4.412600 | −6.725200 | −3.554400 |
| C | 2.048600 | −5.099900 | −1.831600 | C | 2.048600 | −5.099900 | −1.831600 |
| H | 2.539300 | −5.909400 | −2.423800 | H | 2.539300 | −5.909400 | −2.423800 |
| C | 1.664900 | −5.606800 | −0.429600 | C | 1.664900 | −5.606800 | −0.429600 |
| H | 1.592600 | −4.752400 | 0.304600 | H | 1.592600 | −4.752400 | 0.304600 |
| C | 0.384700 | −6.461700 | −0.410900 | C | 0.384700 | −6.461700 | −0.410900 |
| H | 0.663300 | −7.543500 | −0.508500 | H | 0.663300 | −7.543500 | −0.508500 |
| C | −0.650300 | −6.091200 | −1.503000 | C | −0.650300 | −6.091200 | −1.503000 |
| H | −1.243300 | −6.964300 | −1.864400 | H | −1.243300 | −6.964300 | −1.864400 |
| C | 0.874100 | −4.501600 | −2.604100 | C | 0.874100 | −4.501600 | −2.604100 |
| H | 0.407600 | −3.616000 | −2.075400 | H | 0.407600 | −3.616000 | −2.075400 |
| C | 1.164200 | −4.171100 | −4.075800 | C | 1.164200 | −4.171100 | −4.075800 |
| H | 0.467200 | −4.721200 | −4.741600 | H | 0.467200 | −4.721200 | −4.741600 |
| H | 1.074500 | −3.080900 | −4.247600 | H | 1.074500 | −3.080900 | −4.247600 |
| C | −2.993300 | −5.499400 | −1.061900 | C | −2.993300 | −5.499400 | −1.061900 |
| H | −3.043100 | −6.593900 | −1.288300 | H | −3.043100 | −6.593900 | −1.288300 |
| C | −3.663200 | −5.222100 | 0.295500 | C | −3.663200 | −5.222100 | 0.295500 |
| H | −2.998800 | −4.598900 | 0.969000 | H | −2.998800 | −4.598900 | 0.969000 |
| C | −5.067100 | −4.587600 | 0.260500 | C | −5.067100 | −4.587600 | 0.260500 |
| H | −5.771600 | −5.269500 | 0.810200 | H | −5.771600 | −5.269500 | 0.810200 |
| C | −5.655500 | −4.262100 | −1.139000 | C | −5.655500 | −4.262100 | −1.139000 |
| H | −6.710000 | −4.585300 | −1.265900 | H | −6.710000 | −4.585300 | −1.265900 |
| C | −3.544100 | −4.678600 | −2.235200 | C | −3.544100 | −4.678600 | −2.235200 |
| H | −3.321900 | −3.586500 | −2.123400 | H | −3.321900 | −3.586500 | −2.123400 |
| C | −3.113700 | −5.219300 | −3.602600 | C | −3.113700 | −5.219300 | −3.602600 |
| H | −3.582000 | −4.653000 | −4.426400 | H | −3.582000 | −4.653000 | −4.426400 |
| H | −2.003200 | −5.206300 | −3.707800 | H | −2.003200 | −5.206300 | −3.707800 |
| O | −3.090900 | 4.343200 | −0.519900 | O | −3.090900 | 4.343200 | −0.519900 |
| O | −3.234900 | 7.232400 | 0.219900 | O | −3.234900 | 7.232400 | 0.219900 |
| H | −2.962400 | 7.813300 | 0.964600 | H | −2.962400 | 7.813300 | 0.964600 |
| O | −0.345600 | 8.189900 | 0.100300 | O | −0.345600 | 8.189900 | 0.100300 |
| H | 0.347700 | 7.539500 | 0.439500 | H | 0.347700 | 7.539500 | 0.439500 |
| O | 0.603100 | 6.062000 | −1.102100 | O | 0.603100 | 6.062000 | −1.102100 |
| O | −0.873100 | 6.084700 | −2.857100 | O | −0.873100 | 6.084700 | −2.857100 |
| O | −2.784700 | 2.987400 | −3.019200 | O | −2.784700 | 2.987400 | −3.019200 |
| H | −2.284200 | 2.480500 | −2.296000 | H | −2.284200 | 2.480500 | −2.296000 |
| O | 2.312300 | 6.428500 | 1.031800 | O | 2.312300 | 6.428500 | 1.031800 |
| H | 2.160600 | 6.002000 | 1.912300 | H | 2.160600 | 6.002000 | 1.912300 |
| O | 4.351800 | 4.121800 | 1.023600 | O | 4.351800 | 4.121800 | 1.023600 |
| O | 3.843300 | 5.040200 | −2.507200 | O | 3.843300 | 5.040200 | −2.507200 |
| O | 2.401000 | 7.207600 | −3.873200 | O | 2.401000 | 7.207600 | −3.873200 |
| H | 3.364900 | 7.324300 | −3.720200 | H | 3.364900 | 7.324300 | −3.720200 |
| C | −2.632800 | 5.506800 | −1.287000 | C | −2.632800 | 5.506800 | −1.287000 |
| H | −3.501900 | 5.924200 | −1.843500 | H | −3.501900 | 5.924200 | −1.843500 |
| C | −2.075800 | 6.528200 | −0.275000 | C | −2.075800 | 6.528200 | −0.275000 |
| H | −1.576200 | 6.000200 | 0.578500 | H | −1.576200 | 6.000200 | 0.578500 |
| C | −1.136200 | 7.574100 | −0.912400 | C | −1.136200 | 7.574100 | −0.912400 |
| H | −1.712500 | 8.416400 | −1.355600 | H | −1.712500 | 8.416400 | −1.355600 |
| C | −0.181100 | 6.919100 | −1.938500 | C | −0.181100 | 6.919100 | −1.938500 |
| H | 0.409100 | 7.612000 | −2.570300 | H | 0.409100 | 7.612000 | −2.570300 |
| C | −1.545400 | 4.952900 | −2.224700 | C | −1.545400 | 4.952900 | −2.224700 |
| H | −0.777400 | 4.367300 | −1.643700 | H | −0.777400 | 4.367300 | −1.643700 |
| C | −2.069800 | 4.149500 | −3.424500 | C | −2.069800 | 4.149500 | −3.424500 |
| H | −2.802300 | 4.719200 | −4.024400 | H | −2.802300 | 4.719200 | −4.024400 |
| H | −1.224500 | 3.840700 | −4.070100 | H | −1.224500 | 3.840700 | −4.070100 |
| C | 2.029800 | 6.052700 | −1.275300 | C | 2.029800 | 6.052700 | −1.275300 |
| H | 2.405900 | 7.093800 | −1.376100 | H | 2.405900 | 7.093800 | −1.376100 |
| C | 2.482400 | 5.395500 | 0.044500 | C | 2.482400 | 5.395500 | 0.044500 |
| H | 1.824100 | 4.517100 | 0.308600 | H | 1.824100 | 4.517100 | 0.308600 |
| C | 3.979800 | 5.027500 | 0.000600 | C | 3.979800 | 5.027500 | 0.000600 |
| H | 4.584700 | 5.939100 | 0.251400 | H | 4.584700 | 5.939100 | 0.251400 |
| C | 4.452100 | 4.470400 | −1.363200 | C | 4.452100 | 4.470400 | −1.363200 |
| H | 5.537000 | 4.665200 | −1.542400 | H | 5.537000 | 4.665200 | −1.542400 |
| C | 2.394200 | 5.178000 | −2.487300 | C | 2.394200 | 5.178000 | −2.487300 |
| H | 1.931300 | 4.159200 | −2.400300 | H | 1.931300 | 4.159200 | −2.400300 |
| C | 2.085200 | 5.808200 | −3.848900 | C | 2.085200 | 5.808200 | −3.848900 |
| H | 2.641600 | 5.295200 | −4.656600 | H | 2.641600 | 5.295200 | −4.656600 |
| H | 0.992700 | 5.783900 | −4.062300 | H | 0.992700 | 5.783900 | −4.062300 |
| O | 4.192100 | 3.032800 | −1.423400 | O | 4.192100 | 3.032800 | −1.423400 |
| O | 6.681900 | 2.977100 | −0.022900 | O | 6.681900 | 2.977100 | −0.022900 |
| O | 8.244700 | 0.541800 | −0.275400 | O | 8.244700 | 0.541800 | −0.275400 |
| H | 8.072700 | 1.006500 | 0.579100 | H | 8.072700 | 1.006500 | 0.579100 |
| O | 6.239800 | −0.874400 | −1.451700 | O | 6.239800 | −0.874400 | −1.451700 |
| O | 6.167600 | 0.502400 | −3.288800 | O | 6.167600 | 0.502400 | −3.288800 |
| O | 4.330100 | 2.824100 | −4.524100 | O | 4.330100 | 2.824100 | −4.524100 |
| H | 4.002400 | 3.451500 | −3.825000 | H | 4.002400 | 3.451500 | −3.825000 |
| O | 7.083700 | −3.069600 | 0.354900 | O | 7.083700 | −3.069600 | 0.354900 |
| H | 7.408600 | −2.200800 | 0.688100 | H | 7.408600 | −2.200800 | 0.688100 |
| O | 4.377100 | −4.473600 | 0.684100 | O | 4.377100 | −4.473600 | 0.684100 |
| O | 4.958400 | −3.992600 | −2.909000 | O | 4.958400 | −3.992600 | −2.909000 |
| O | 7.181500 | −3.006100 | −4.407600 | O | 7.181500 | −3.006100 | −4.407600 |
| H | 7.087200 | −3.963500 | −4.198400 | H | 7.087200 | −3.963500 | −4.198400 |
| C | 5.370600 | 2.327000 | −1.969200 | C | 5.370600 | 2.327000 | −1.969200 |
| H | 5.942100 | 3.046200 | −2.601900 | H | 5.942100 | 3.046200 | −2.601900 |
| C | 6.200000 | 1.851700 | −0.757600 | C | 6.200000 | 1.851700 | −0.757600 |
| H | 5.592100 | 1.186300 | −0.082600 | H | 5.592100 | 1.186300 | −0.082600 |
| C | 7.455900 | 1.143700 | −1.306000 | C | 7.455900 | 1.143700 | −1.306000 |
| H | 8.125500 | 1.859500 | −1.837100 | H | 8.125500 | 1.859500 | −1.837100 |
| C | 7.021100 | −0.004600 | −2.255400 | C | 7.021100 | −0.004600 | −2.255400 |
| H | 7.843800 | −0.502200 | −2.801300 | H | 7.843800 | −0.502200 | −2.801300 |
| C | 4.928400 | 1.122700 | −2.813000 | C | 4.928400 | 1.122700 | −2.813000 |
| H | 4.380500 | 0.355700 | −2.197400 | H | 4.380500 | 0.355700 | −2.197400 |
| C | 4.158600 | 1.470900 | −4.093600 | C | 4.158600 | 1.470900 | −4.093600 |
| H | 4.549600 | 0.886300 | −4.951200 | H | 4.549600 | 0.886300 | −4.951200 |
| H | 3.075200 | 1.273600 | −3.955000 | H | 3.075200 | 1.273600 | −3.955000 |
| C | 6.279100 | −2.290100 | −1.751000 | C | 6.279100 | −2.290100 | −1.751000 |
| H | 7.317200 | −2.597600 | −2.014300 | H | 7.317200 | −2.597600 | −2.014300 |
| C | 5.861600 | −2.862400 | −0.378900 | C | 5.861600 | −2.862400 | −0.378900 |
| H | 5.208500 | −2.125200 | 0.174300 | H | 5.208500 | −2.125200 | 0.174300 |
| C | 5.217200 | −4.255100 | −0.434800 | C | 5.217200 | −4.255100 | −0.434800 |
| H | 6.016400 | −5.033000 | −0.304500 | H | 6.016400 | −5.033000 | −0.304500 |
| C | 4.416800 | −4.549100 | −1.726200 | C | 4.416800 | −4.549100 | −1.726200 |
| H | 4.382700 | −5.639500 | −1.963100 | H | 4.382700 | −5.639500 | −1.963100 |
| C | 5.295400 | −2.577800 | −2.895100 | C | 5.295400 | −2.577800 | −2.895100 |
| H | 4.354000 | −1.983700 | −2.770300 | H | 4.354000 | −1.983700 | −2.770300 |
| C | 5.899800 | −2.374300 | −4.291400 | C | 5.899800 | −2.374300 | −4.291400 |
| H | 5.229300 | −2.776600 | −5.073200 | H | 5.229300 | −2.776600 | −5.073200 |
| H | 6.115200 | −1.299800 | −4.484300 | H | 6.115200 | −1.299800 | −4.484300 |
| H | −6.761900 | −2.594700 | 1.019200 | H | −6.761900 | −2.594700 | 1.019200 |
| H | −3.379400 | 2.333400 | 2.146600 | H | −3.379400 | 2.333400 | 2.146600 |
| H | −3.984200 | 1.818100 | −0.566200 | H | −3.984200 | 1.818100 | −0.566200 |
| H | −4.382100 | −2.732900 | 0.752900 | H | −4.382100 | −2.732900 | 0.752900 |
| H | 3.722300 | 3.300100 | 1.054400 | H | 3.722300 | 3.300100 | 1.054400 |
| H | 5.938200 | 3.318700 | 0.584100 | H | 5.938200 | 3.318700 | 0.584100 |
| H | 3.693800 | −3.707400 | 0.799600 | H | 3.693800 | −3.707400 | 0.799600 |
| I | 1.564100 | −0.295900 | 4.951000 | I | 1.564100 | −0.295900 | 4.951000 |
| I | 1.894300 | −3.326600 | 2.575000 | I | 1.894300 | −3.326600 | 2.575000 |
| I | 4.041500 | −0.059500 | 1.906900 | I | 4.041500 | −0.059500 | 1.906900 |
| I | −1.715400 | 1.544800 | 4.067900 | I | −1.715400 | 1.544800 | 4.067900 |
| I | 2.389100 | −2.091300 | −0.994000 | I | 2.389100 | −2.091300 | −0.994000 |
| I | 1.727700 | 3.020000 | 2.854900 | I | 1.727700 | 3.020000 | 2.854900 |
| I | −0.830900 | 0.111000 | −2.118600 | I | −0.830900 | 0.111000 | −2.118600 |
| I | −1.650500 | −2.360100 | 3.674200 | I | −1.650500 | −2.360100 | 3.674200 |
| I | −1.603400 | 2.973900 | 0.515800 | I | −1.603400 | 2.973900 | 0.515800 |
| I | −1.094300 | −3.231800 | −0.073900 | I | −1.094300 | −3.231800 | −0.073900 |
| I | 2.434700 | 1.975700 | −0.838500 | I | 2.434700 | 1.975700 | −0.838500 |
| I | −3.397400 | −0.218100 | 1.021500 | I | −3.397400 | −0.218100 | 1.021500 |
| B | 1.050000 | −1.531500 | 1.885800 | B | 1.050000 | −1.531500 | 1.885800 |
| B | 0.861400 | −0.202200 | 2.990400 | B | 0.861400 | −0.202200 | 2.990400 |
| B | 1.957100 | −0.083000 | 1.645400 | B | 1.957100 | −0.083000 | 1.645400 |
| B | 0.932800 | 1.254800 | 2.044500 | B | 0.932800 | 1.254800 | 2.044500 |
| B | −0.286500 | −1.515000 | 0.788300 | B | −0.286500 | −1.515000 | 0.788300 |
| B | 1.251700 | −0.886300 | 0.283500 | B | 1.251700 | −0.886300 | 0.283500 |
| B | −0.523800 | −1.092900 | 2.453300 | B | −0.523800 | −1.092900 | 2.453300 |
| B | −0.406400 | 1.312500 | 0.935900 | B | −0.406400 | 1.312500 | 0.935900 |
| B | −0.211500 | −0.044300 | −0.120000 | B | −0.211500 | −0.044300 | −0.120000 |
| B | 1.177500 | 0.839100 | 0.389800 | B | 1.177500 | 0.839100 | 0.389800 |
| B | −0.597600 | 0.639600 | 2.539600 | B | −0.597600 | 0.639600 | 2.539600 |
| B | −1.312700 | −0.183600 | 1.203300 | B | −1.312700 | −0.183600 | 1.203300 |

TABLE 4
Cartesian coordinates (Å) of the optimized (PM6) structure
of the [(HP-γ-CD)(B₁₂I₁₂)]²⁻ complex.

| | | | | | | | |
|---|---|---|---|---|---|---|---|
| O | −4.742300 | −4.095800 | −1.283100 | O | −4.742300 | −4.095800 | −1.283100 |
| O | −6.929500 | −3.964000 | 0.633000 | O | −6.929500 | −3.964000 | 0.633000 |
| H | −6.090800 | −4.442200 | 0.943500 | H | −6.090800 | −4.442200 | 0.943500 |
| O | −8.068500 | −1.337600 | 0.549400 | O | −8.068500 | −1.337600 | 0.549400 |
| H | −8.198600 | −1.946900 | 1.322200 | H | −8.198600 | −1.946900 | 1.322200 |
| O | −6.324300 | −0.422400 | −1.602100 | O | −6.324300 | −0.422400 | −1.602100 |
| O | −7.228600 | −1.981100 | −2.939900 | O | −7.228600 | −1.981100 | −2.939900 |
| O | −5.585500 | −4.833700 | −4.130600 | O | −5.585500 | −4.833700 | −4.130600 |
| H | −4.602100 | −4.895800 | −3.915700 | H | −4.602100 | −4.895800 | −3.915700 |
| O | −5.186700 | −0.513200 | 1.021600 | O | −5.186700 | −0.513200 | 1.021600 |
| H | −6.109400 | −0.888700 | 1.182300 | H | −6.109400 | −0.888700 | 1.182300 |
| O | −4.065800 | 2.099600 | 1.821800 | O | −4.065800 | 2.099600 | 1.821800 |
| O | −6.289000 | 3.160600 | −0.778800 | O | −6.289000 | 3.160600 | −0.778800 |
| O | −8.729800 | 1.856400 | −2.077300 | O | −8.729800 | 1.856400 | −2.077300 |
| C | −6.094800 | −3.727600 | −1.638800 | C | −6.094800 | −3.727600 | −1.638800 |
| H | −6.711300 | −4.622700 | −1.863700 | H | −6.711300 | −4.622700 | −1.863700 |
| C | −6.552000 | −3.000900 | −0.355600 | C | −6.552000 | −3.000900 | −0.355600 |
| H | −5.705400 | −2.371400 | 0.042400 | H | −5.705400 | −2.371400 | 0.042400 |
| C | −7.798800 | −2.145100 | −0.612200 | C | −7.798800 | −2.145100 | −0.612200 |
| H | −8.695000 | −2.773200 | −0.813000 | H | −8.695000 | −2.773200 | −0.813000 |
| C | −7.523700 | −1.184900 | −1.803200 | C | −7.523700 | −1.184900 | −1.803200 |
| H | −8.379700 | −0.541900 | −2.094600 | H | −8.379700 | −0.541900 | −2.094600 |
| C | −5.997300 | −2.771300 | −2.844100 | C | −5.997300 | −2.771300 | −2.844100 |
| H | −5.142200 | −2.066100 | −2.709700 | H | −5.142200 | −2.066100 | −2.709700 |
| C | −5.988200 | −3.471300 | −4.217900 | C | −5.988200 | −3.471300 | −4.217900 |
| H | −7.018200 | −3.527300 | −4.628100 | H | −7.018200 | −3.527300 | −4.628100 |
| H | −5.338300 | −2.932000 | −4.929200 | H | −5.338300 | −2.932000 | −4.929200 |
| C | −6.453700 | 0.727300 | −0.743400 | C | −6.453700 | 0.727300 | −0.743400 |
| H | −7.439400 | 0.715900 | −0.213000 | H | −7.439400 | 0.715900 | −0.213000 |
| C | −5.251400 | 0.684500 | 0.210500 | C | −5.251400 | 0.684500 | 0.210500 |
| C | −5.313700 | 1.919100 | 1.135800 | C | −5.313700 | 1.919100 | 1.135800 |
| H | −6.143300 | 1.862300 | 1.864300 | H | −6.143300 | 1.862300 | 1.864300 |
| C | −5.349000 | 3.203000 | 0.282500 | C | −5.349000 | 3.203000 | 0.282500 |
| H | −5.615300 | 4.139800 | 0.822500 | H | −5.615300 | 4.139800 | 0.822500 |
| C | −6.287700 | 1.979700 | −1.632300 | C | −6.287700 | 1.979700 | −1.632300 |
| H | −5.294600 | 1.958100 | −2.171100 | H | −5.294600 | 1.958100 | −2.171100 |
| C | −7.437600 | 2.197400 | −2.617000 | C | −7.437600 | 2.197400 | −2.617000 |
| H | −7.445800 | 3.236100 | −2.990200 | H | −7.445800 | 3.236100 | −2.990200 |
| H | −7.357600 | 1.481700 | −3.461900 | H | −7.357600 | 1.481700 | −3.461900 |
| O | 4.074600 | −3.223400 | −1.271000 | O | 4.074600 | −3.223400 | −1.271000 |
| O | 4.184600 | −5.784200 | 0.177600 | O | 4.184600 | −5.784200 | 0.177600 |
| H | 4.661800 | −5.175500 | 0.825400 | H | 4.661800 | −5.175500 | 0.825400 |
| O | 1.238500 | −6.525600 | 0.774400 | O | 1.238500 | −6.525600 | 0.774400 |
| H | 0.917100 | −5.659700 | 1.182900 | H | 0.917100 | −5.659700 | 1.182900 |
| O | −0.195900 | −5.270700 | −0.976400 | O | −0.195900 | −5.270700 | −0.976400 |
| O | 1.374600 | −5.344200 | −2.683200 | O | 1.374600 | −5.344200 | −2.683200 |
| O | 3.793100 | −3.767500 | −4.260600 | O | 3.793100 | −3.767500 | −4.260600 |
| H | 4.395600 | −3.258700 | −3.651100 | H | 4.395600 | −3.258700 | −3.651100 |
| O | −2.048300 | −7.422300 | 0.393300 | O | −2.048300 | −7.422300 | 0.393300 |
| H | −1.129700 | −7.519800 | 0.754600 | H | −1.129700 | −7.519800 | 0.754600 |
| O | −4.202400 | −4.924100 | 0.935500 | O | −4.202400 | −4.924100 | 0.935500 |
| H | −3.746400 | −4.030100 | 0.789800 | H | −3.746400 | −4.030100 | 0.789800 |
| O | −3.488500 | −5.515500 | −2.512500 | O | −3.488500 | −5.515500 | −2.512500 |
| O | −1.879300 | −5.364800 | −4.628500 | O | −1.879300 | −5.364800 | −4.628500 |
| C | 3.341700 | −4.471200 | −1.616000 | C | 3.341700 | −4.471200 | −1.616000 |
| H | 4.033400 | −5.122400 | −2.203400 | H | 4.033400 | −5.122400 | −2.203400 |
| C | 2.983300 | −5.137700 | −0.272700 | C | 2.983300 | −5.137700 | −0.272700 |
| H | 2.646700 | −4.375500 | 0.485700 | H | 2.646700 | −4.375500 | 0.485700 |
| C | 1.953600 | −6.273700 | −0.428000 | C | 1.953600 | −6.273700 | −0.428000 |
| H | 2.493300 | −7.240200 | −0.603600 | H | 2.493300 | −7.240200 | −0.603600 |
| C | 0.917400 | −6.041900 | −1.555500 | C | 0.917400 | −6.041900 | −1.555500 |
| H | 0.507200 | −6.988500 | −1.990300 | H | 0.507200 | −6.988500 | −1.990300 |
| C | 2.117200 | −4.100600 | −2.452200 | C | 2.117200 | −4.100600 | −2.452200 |
| H | 1.417900 | −3.392600 | −1.911700 | H | 1.417900 | −3.392600 | −1.911700 |
| C | 2.429200 | −3.594200 | −3.867500 | C | 2.429200 | −3.594200 | −3.867500 |
| H | 1.874000 | −4.186600 | −4.623600 | H | 1.874000 | −4.186600 | −4.623600 |
| H | 2.165900 | −2.521600 | −3.955200 | H | 2.165900 | −2.521600 | −3.955200 |
| C | −1.510500 | −5.823900 | −1.313600 | C | −1.510500 | −5.823900 | −1.313600 |
| H | −1.356300 | −6.807200 | −1.827900 | H | −1.356300 | −6.807200 | −1.827900 |
| C | −2.251500 | −6.044700 | 0.018700 | C | −2.251500 | −6.044700 | 0.018700 |
| H | −1.840000 | −5.384600 | 0.838000 | H | −1.840000 | −5.384600 | 0.838000 |
| C | −3.785500 | −5.878500 | −0.036700 | C | −3.785500 | −5.878500 | −0.036700 |
| H | −4.260600 | −6.827200 | 0.324800 | H | −4.260600 | −6.827200 | 0.324800 |
| C | −4.393400 | −5.476400 | −1.414200 | C | −4.393400 | −5.476400 | −1.414200 |
| H | −5.237000 | −6.112600 | −1.749100 | H | −5.237000 | −6.112600 | −1.749100 |
| C | −2.237900 | −4.819200 | −2.217800 | C | −2.237900 | −4.819200 | −2.217800 |
| H | −2.463100 | −3.860400 | −1.670200 | H | −2.463100 | −3.860400 | −1.670200 |
| C | −1.567100 | −4.473500 | −3.547100 | C | −1.567100 | −4.473500 | −3.547100 |
| H | −1.963000 | −3.514700 | −3.942200 | H | −1.963000 | −3.514700 | −3.942200 |
| H | −0.466900 | −4.392300 | −3.403400 | H | −0.466900 | −4.392300 | −3.403400 |
| O | −3.988000 | 3.304200 | −0.252700 | O | −3.988000 | 3.304200 | −0.252700 |
| O | −4.807200 | 6.059100 | 0.522200 | O | −4.807200 | 6.059100 | 0.522200 |
| H | −4.674600 | 6.685700 | 1.267400 | H | −4.674600 | 6.685700 | 1.267400 |
| O | −2.273900 | 7.731500 | 0.351600 | O | −2.273900 | 7.731500 | 0.351600 |
| H | −1.425100 | 7.292700 | 0.677800 | H | −1.425100 | 7.292700 | 0.677800 |
| O | −0.799900 | 5.935100 | −0.850300 | O | −0.799900 | 5.935100 | −0.850300 |
| O | −2.247100 | 5.532400 | −2.586200 | O | −2.247100 | 5.532400 | −2.586200 |
| O | −3.372300 | 2.070100 | −2.709100 | O | −3.372300 | 2.070100 | −2.709100 |
| H | −2.782600 | 1.694600 | −1.971800 | H | −2.782600 | 1.694600 | −1.971800 |
| O | 0.830600 | 6.815000 | 1.209900 | O | 0.830600 | 6.815000 | 1.209900 |
| H | 0.898500 | 6.432900 | 2.118100 | H | 0.898500 | 6.432900 | 2.118100 |
| O | 3.319300 | 5.023900 | 1.238000 | O | 3.319300 | 5.023900 | 1.238000 |
| O | 2.564000 | 5.612700 | −2.321500 | O | 2.564000 | 5.612700 | −2.321500 |
| O | 0.686400 | 7.482100 | −3.527200 | O | 0.686400 | 7.482100 | −3.527200 |
| C | −3.810500 | 4.547600 | −1.011400 | C | −3.810500 | 4.547600 | −1.011400 |
| H | −4.748300 | 4.748200 | −1.576000 | H | −4.748300 | 4.748200 | −1.576000 |
| C | −3.516300 | 5.670400 | 0.003600 | C | −3.516300 | 5.670400 | 0.003600 |
| H | −2.885500 | 5.284300 | 0.845900 | H | −2.885500 | 5.284300 | 0.845900 |
| C | −2.885300 | 6.918400 | −0.648000 | C | −2.885300 | 6.918400 | −0.648000 |
| H | −3.664200 | 7.576800 | −1.093400 | H | −3.664200 | 7.576800 | −1.093400 |
| C | −1.799900 | 6.530900 | −1.679800 | C | −1.799900 | 6.530900 | −1.679800 |
| H | −1.440600 | 7.361900 | −2.322700 | H | −1.440600 | 7.361900 | −2.322700 |
| C | −2.612100 | 4.275300 | −1.939600 | C | −2.612100 | 4.275300 | −1.939600 |
| H | −1.722400 | 3.913400 | −1.350500 | H | −1.722400 | 3.913400 | −1.350500 |
| C | −2.912700 | 3.350100 | −3.128900 | C | −2.912700 | 3.350100 | −3.128900 |
| H | −3.732500 | 3.735400 | −3.761600 | H | −3.732500 | 3.735400 | −3.761600 |
| H | −2.001100 | 3.217200 | −3.743500 | H | −2.001100 | 3.217200 | −3.743500 |
| C | 0.581600 | 6.283500 | −1.067200 | C | 0.581600 | 6.283500 | −1.067200 |
| H | 0.682700 | 7.382200 | −1.207300 | H | 0.682700 | 7.382200 | −1.207300 |
| C | 1.206200 | 5.801700 | 0.256200 | C | 1.206200 | 5.801700 | 0.256200 |
| H | 0.766600 | 4.813300 | 0.579500 | H | 0.766600 | 4.813300 | 0.579500 |
| C | 2.745400 | 5.770900 | 0.179300 | C | 2.745400 | 5.770900 | 0.179300 |
| H | 3.140400 | 6.804200 | 0.369400 | H | 3.140400 | 6.804200 | 0.369400 |
| C | 3.300900 | 5.264800 | −1.172100 | C | 3.300900 | 5.264800 | −1.172100 |
| H | 4.314900 | 5.680800 | −1.393400 | H | 4.314900 | 5.680800 | −1.393400 |
| C | 1.116600 | 5.490600 | −2.273200 | C | 1.116600 | 5.490600 | −2.273200 |
| H | 0.856000 | 4.402700 | −2.179900 | H | 0.856000 | 4.402700 | −2.179900 |
| C | 0.676500 | 6.036300 | −3.636300 | C | 0.676500 | 6.036300 | −3.636300 |
| H | 1.404900 | 5.763200 | −4.423600 | H | 1.404900 | 5.763200 | −4.423600 |
| H | −0.338800 | 5.669400 | −3.889300 | H | −0.338800 | 5.669400 | −3.889300 |
| O | 3.368500 | 3.800700 | −1.150100 | O | 3.368500 | 3.800700 | −1.150100 |
| O | 5.823100 | 4.563900 | 0.026300 | O | 5.823100 | 4.563900 | 0.026300 |
| H | 5.149400 | 4.741100 | 0.758100 | H | 5.149400 | 4.741100 | 0.758100 |
| O | 7.851000 | 2.309500 | 0.098300 | O | 7.851000 | 2.309500 | 0.098300 |
| H | 7.426000 | 1.539000 | 0.579600 | H | 7.426000 | 1.539000 | 0.579600 |
| O | 6.407600 | 0.513000 | −0.944100 | O | 6.407600 | 0.513000 | −0.944100 |
| O | 5.816000 | 1.619500 | −2.873400 | O | 5.816000 | 1.619500 | −2.873400 |
| O | 3.403600 | 3.266000 | −4.182200 | O | 3.403600 | 3.266000 | −4.182200 |
| H | 2.978900 | 3.876200 | −3.522200 | H | 2.978900 | 3.876200 | −3.522200 |
| O | 7.336200 | −1.225500 | 1.169800 | O | 7.336200 | −1.225500 | 1.169800 |
| H | 6.952100 | −1.358700 | 2.070000 | H | 6.952100 | −1.358700 | 2.070000 |
| O | 5.315200 | −3.470300 | 1.149300 | O | 5.315200 | −3.470300 | 1.149300 |
| O | 6.061700 | −2.838900 | −2.398700 | O | 6.061700 | −2.838900 | −2.398700 |
| O | 8.608300 | −1.531000 | −3.227400 | O | 8.608300 | −1.531000 | −3.227400 |
| C | 4.666500 | 3.332200 | −1.679900 | C | 4.666500 | 3.332200 | −1.679900 |
| H | 5.024400 | 4.085000 | −2.421100 | H | 5.024400 | 4.085000 | −2.421100 |
| C | 5.624500 | 3.236700 | −0.469200 | C | 5.624500 | 3.236700 | −0.469200 |
| H | 5.201000 | 2.580700 | 0.335300 | H | 5.201000 | 2.580700 | 0.335300 |
| C | 6.998600 | 2.740000 | −0.963800 | C | 6.998600 | 2.740000 | −0.963800 |
| H | 7.576000 | 3.561700 | −1.443400 | H | 7.576000 | 3.561700 | −1.443400 |
| C | 6.825000 | 1.502700 | −1.890800 | C | 6.825000 | 1.502700 | −1.890800 |
| H | 7.734600 | 1.236200 | −2.467900 | H | 7.734600 | 1.236200 | −2.467900 |
| C | 4.486900 | 1.968700 | −2.359500 | C | 4.486900 | 1.968700 | −2.359500 |
| H | 4.187400 | 1.159700 | −1.634200 | H | 4.187400 | 1.159700 | −1.634200 |
| C | 3.593100 | 1.969400 | −3.606300 | C | 3.593100 | 1.969400 | −3.606300 |
| H | 4.073600 | 1.399900 | −4.427000 | H | 4.073600 | 1.399900 | −4.427000 |
| H | 2.598900 | 1.533300 | −3.367200 | H | 2.598900 | 1.533300 | −3.367200 |
| C | 6.861500 | −0.851100 | −1.096900 | C | 6.861500 | −0.851100 | −1.096900 |
| H | 7.983900 | −0.872100 | −1.080900 | H | 7.983900 | −0.872100 | −1.080900 |
| C | 6.305700 | −1.473200 | 0.190500 | C | 6.305700 | −1.473200 | 0.190500 |
| H | 5.359300 | −0.942100 | 0.519700 | H | 5.359300 | −0.942100 | 0.519700 |
| C | 6.119600 | −2.994900 | 0.083600 | C | 6.119600 | −2.994900 | 0.083600 |
| H | 7.101300 | −3.508600 | 0.250600 | H | 7.101300 | −3.508600 | 0.250600 |
| C | 5.521500 | −3.461400 | −1.258000 | C | 5.521500 | −3.461400 | −1.258000 |
| H | 5.718300 | −4.544200 | −1.456900 | H | 5.718300 | −4.544200 | −1.456900 |
| C | 6.290100 | −1.406400 | −2.410200 | C | 6.290100 | −1.406400 | −2.410200 |
| H | 5.301700 | −0.922700 | −2.624500 | H | 5.301700 | −0.922700 | −2.624500 |
| C | 7.244100 | −1.248500 | −3.611300 | C | 7.244100 | −1.248500 | −3.611300 |
| H | 7.024100 | −2.037900 | −4.361900 | H | 7.024100 | −2.037900 | −4.361900 |
| H | 7.139000 | −0.248900 | −4.067100 | H | 7.139000 | −0.248900 | −4.067100 |
| I | 1.581900 | −0.034400 | 5.262200 | I | 1.581900 | −0.034400 | 5.262200 |
| I | 2.667700 | −2.890800 | 2.897800 | I | 2.667700 | −2.890800 | 2.897800 |
| I | 3.964100 | 0.840900 | 2.260700 | I | 3.964100 | 0.840900 | 2.260700 |
| I | −2.045900 | 0.907400 | 4.334200 | I | −2.045900 | 0.907400 | 4.334200 |
| I | 2.943100 | −1.502700 | −0.687100 | I | 2.943100 | −1.502700 | −0.687100 |
| I | 0.936200 | 3.243500 | 3.137500 | I | 0.936200 | 3.243500 | 3.137500 |
| I | −0.724300 | −0.208600 | −1.848600 | I | −0.724300 | −0.208600 | −1.848600 |
| I | −0.992300 | −2.841700 | 3.926300 | I | −0.992300 | −2.841700 | 3.926300 |
| I | −2.236900 | 2.348900 | 0.791400 | I | −2.236900 | 2.348900 | 0.791400 |
| I | −0.147300 | −3.488000 | 0.142500 | I | −0.147300 | −3.488000 | 0.142500 |
| I | 1.896600 | 2.425300 | −0.502300 | I | 1.896600 | 2.425300 | −0.502300 |
| I | −3.183000 | −1.203300 | 1.215400 | I | −3.183000 | −1.203300 | 1.215400 |
| B | 1.441700 | −1.339200 | 2.187800 | B | 1.441700 | −1.339200 | 2.187800 |
| B | 0.903700 | −0.111100 | 3.291600 | B | 0.903700 | −0.111100 | 3.291600 |
| B | 1.956200 | 0.290300 | 1.965400 | B | 1.956200 | 0.290300 | 1.965400 |
| B | 0.619600 | 1.323000 | 2.353600 | B | 0.619600 | 1.323000 | 2.353600 |
| B | 0.166300 | −1.656100 | 1.071800 | B | 0.166300 | −1.656100 | 1.071800 |
| B | 1.504700 | −0.657900 | 0.587400 | B | 1.504700 | −0.657900 | 0.587400 |
| B | −0.203500 | −1.321600 | 2.730100 | B | −0.203500 | −1.321600 | 2.730100 |
| B | −0.672100 | 1.037600 | 1.221700 | B | −0.672100 | 1.037600 | 1.221700 |
| B | −0.121800 | −0.215800 | 0.164200 | B | −0.121800 | −0.215800 | 0.164200 |
| B | 0.984800 | 0.989200 | 0.702200 | B | 0.984800 | 0.989200 | 0.702200 |
| B | −0.714000 | 0.332800 | 2.817700 | B | −0.714000 | 0.332800 | 2.817700 |
| B | −1.179300 | −0.638600 | 1.466000 | B | −1.179300 | −0.638600 | 1.466000 |
| C | −9.258300 | 2.832700 | −1.144900 | C | −9.258300 | 2.832700 | −1.144900 |
| C | −10.279700 | 2.035800 | −0.310200 | C | −10.279700 | 2.035800 | −0.310200 |
| H | −9.752900 | 3.624700 | −1.733100 | H | −9.752900 | 3.624700 | −1.733100 |
| H | −8.441500 | 3.270900 | −0.541300 | H | −8.441500 | 3.270900 | −0.541300 |
| C | −9.653700 | 1.360500 | 0.898200 | C | −9.653700 | 1.360500 | 0.898200 |
| H | −10.826400 | 1.310600 | −0.958800 | H | −10.826400 | 1.310600 | −0.958800 |
| H | −10.388400 | 0.765400 | 1.451000 | H | −10.388400 | 0.765400 | 1.451000 |
| H | −8.847500 | 0.665600 | 0.575800 | H | −8.847500 | 0.665600 | 0.575800 |
| H | −9.203600 | 2.070900 | 1.597800 | H | −9.203600 | 2.070900 | 1.597800 |
| O | −11.327700 | 2.937800 | 0.092600 | O | −11.327700 | 2.937800 | 0.092600 |
| H | −11.017400 | 3.545600 | 0.788300 | H | −11.017400 | 3.545600 | 0.788300 |
| C | −1.082700 | −6.560300 | −4.720900 | C | −1.082700 | −6.560300 | −4.720900 |
| C | −1.894700 | −7.723400 | −4.127900 | C | −1.894700 | −7.723400 | −4.127900 |
| H | −0.104300 | −6.445000 | −4.211500 | H | −0.104300 | −6.445000 | −4.211500 |
| H | −0.918700 | −6.660800 | −5.807800 | H | −0.918700 | −6.660800 | −5.807800 |
| C | −2.284700 | −8.787200 | −5.145600 | C | −2.284700 | −8.787200 | −5.145600 |
| H | −2.789300 | −7.322800 | −3.584400 | H | −2.789300 | −7.322800 | −3.584400 |
| H | −2.887100 | −9.580100 | −4.691500 | H | −2.887100 | −9.580100 | −4.691500 |
| H | −2.879100 | −8.349800 | −5.957800 | H | −2.879100 | −8.349800 | −5.957800 |
| H | −1.399200 | −9.257600 | −5.589400 | H | −1.399200 | −9.257600 | −5.589400 |
| O | −0.991000 | −8.301300 | −3.147600 | O | −0.991000 | −8.301300 | −3.147600 |
| H | −1.417000 | −9.041700 | −2.677700 | H | −1.417000 | −9.041700 | −2.677700 |
| C | 9.571500 | −0.473000 | −3.406700 | C | 9.571500 | −0.473000 | −3.406700 |
| C | 10.051400 | −0.025700 | −2.009700 | C | 10.051400 | −0.025700 | −2.009700 |
| H | 9.163100 | 0.379600 | −3.977500 | H | 9.163100 | 0.379600 | −3.977500 |
| H | 10.372000 | −0.957000 | −3.993600 | H | 10.372000 | −0.957000 | −3.993600 |
| C | 11.546700 | −0.214300 | −1.791800 | C | 11.546700 | −0.214300 | −1.791800 |
| H | 9.451500 | −0.555000 | −1.218800 | H | 9.451500 | −0.555000 | −1.218800 |
| H | 11.838400 | 0.116600 | −0.787600 | H | 11.838400 | 0.116600 | −0.787600 |
| H | 11.839600 | −1.264200 | −1.896900 | H | 11.839600 | −1.264200 | −1.896900 |
| H | 12.127800 | 0.384400 | −2.503300 | H | 12.127800 | 0.384400 | −2.503300 |
| O | 9.758400 | 1.384800 | −1.962800 | O | 9.758400 | 1.384800 | −1.962800 |
| H | 9.507000 | 1.671800 | −1.042700 | H | 9.507000 | 1.671800 | −1.042700 |
| C | −0.190800 | 8.163400 | −4.452100 | C | −0.190800 | 8.163400 | −4.452100 |
| C | −0.488800 | 9.488700 | −3.729700 | C | −0.488800 | 9.488700 | −3.729700 |
| H | −1.118800 | 7.586200 | −4.628500 | H | −1.118800 | 7.586200 | −4.628500 |
| H | 0.359000 | 8.296900 | −5.397200 | H | 0.359000 | 8.296900 | −5.397200 |
| C | −0.510800 | 10.689700 | −4.667400 | C | −0.510800 | 10.689700 | −4.667400 |
| H | 0.227600 | 9.632500 | −2.886300 | H | 0.227600 | 9.632500 | −2.886300 |
| H | −0.748400 | 11.611700 | −4.125800 | H | −0.748400 | 11.611700 | −4.125800 |
| H | 0.455400 | 10.832600 | −5.163200 | H | 0.455400 | 10.832600 | −5.163200 |
| H | −1.279100 | 10.569300 | −5.442200 | H | 1.279100 | 10.569300 | −5.442200 |
| O | −1.817300 | 9.301900 | −3.188300 | O | −1.817300 | 9.301900 | −3.188300 |
| H | −1.955400 | 9.863000 | −2.401900 | H | −1.955400 | 9.863000 | −2.401900 |
| H | −3.811800 | 1.236000 | 2.377800 | H | −3.811800 | 1.236000 | 2.377800 |
| H | −4.273500 | 0.658600 | −0.364500 | H | −4.273500 | 0.658600 | −0.364500 |
| H | 2.927900 | 4.072100 | 1.284300 | H | 2.927900 | 4.072100 | 1.284300 |
| H | 4.418500 | −2.945700 | 1.193500 | H | 4.418500 | −2.945700 | 1.193500 |

Although the X-ray diffraction data support the interaction of γ-CD with B₁₂I₁₂²⁻, and suggest that they may form a 2:1 complex in the solid state, gas-phase experiments and solution-phase thermodynamic measurements indicate that the 1:1 complex predominates in solution. To appropriately avoid deviation from ideal-solution behavior, the thermodynamic measurements are performed on relatively dilute solutions (<0.5 mM). Although Na₂B₁₂I₁₂ and γ-CD are both highly water-soluble, PBS solutions containing >6 mM of each species were observed to form an insoluble gel. Addition of PBS to dilute the mixture below this concentration produces fluid solutions. As depicted in FIG. 3, Na₂B₁₂I₁₂ alone does not induce hemolysis at concentrations below 6 mM. Although the chaotropism-driven complexation of B₁₂I₁₂²⁻ by γ-CD may well prevent the physical interaction with cells that leads to hemolysis, the low solubility of the complex prevented investigation of this effect.

Complexes of Na₂B₁₂I₁₂ and Substituted γ-Cyclodextrin

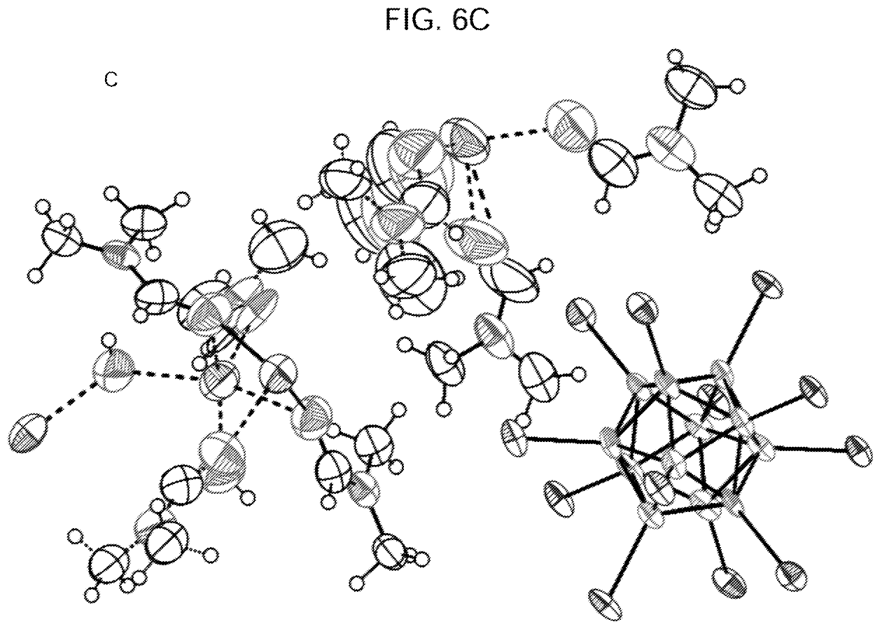

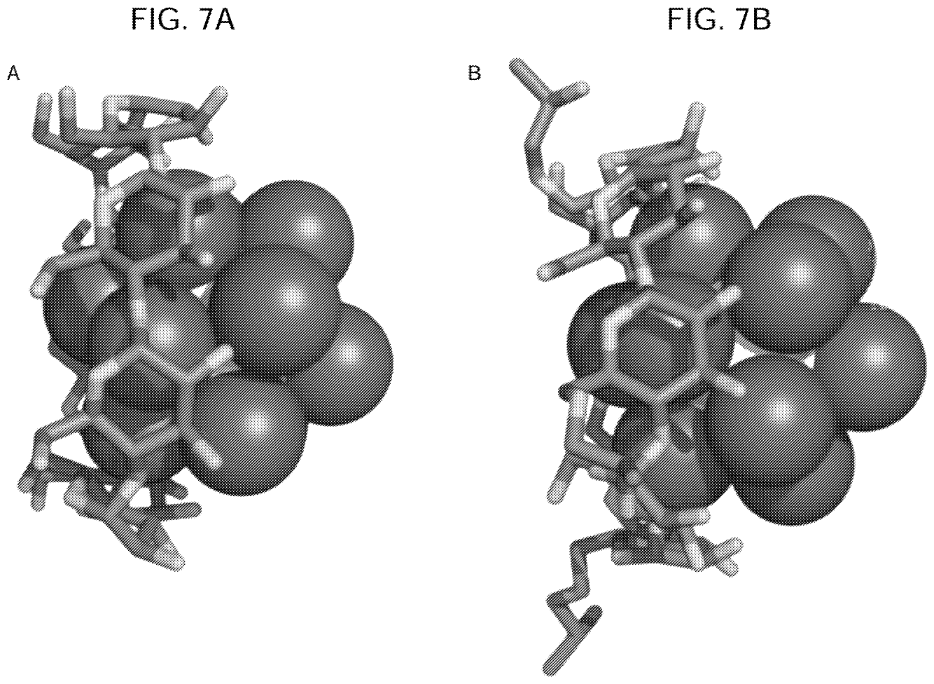

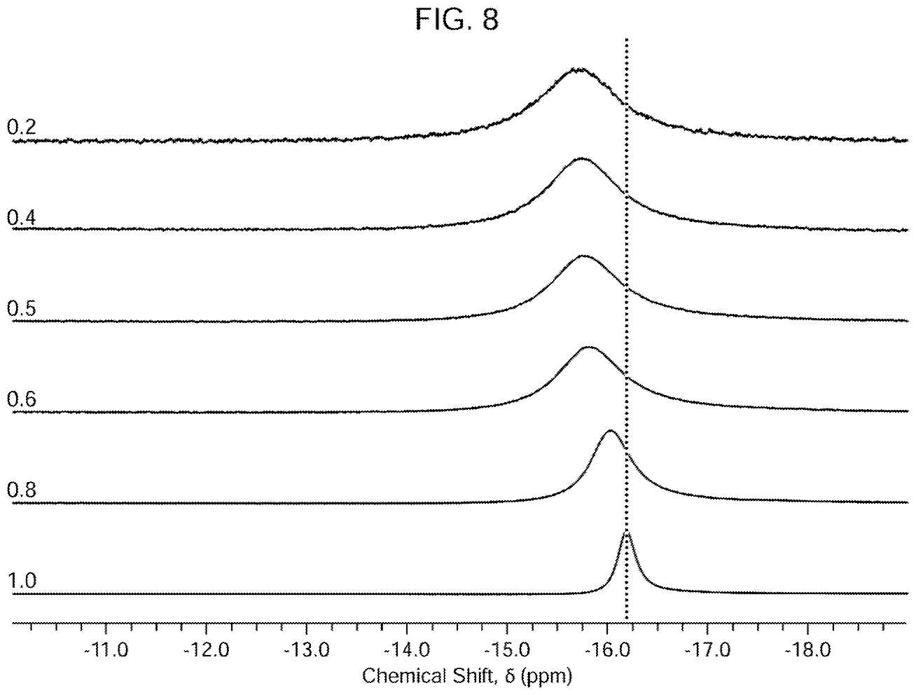

2-hydroxypropyl-γ-CD (HP-γ-CD) is more soluble than γ-CD. Semi-empirical (PM6) geometry optimization calculations predicted that the hydroxypropyl groups would not significantly perturb the host-guest interaction, as compared to unfunctionalized γ-CD (FIG. 7). ¹¹B NMR spectroscopic experiments show that addition of HP-γ-CD to solutions of Na₂B₁₂I₁₂ produces a shift in the resonance for the cluster (FIG. 8). The method of continuous variation permitted the stoichiometry of the complexation between HP-γ-CD and B₁₂I₁₂²⁻ to be determined, confirming that the two form a 1:1 complex in PBS (FIG. 9). Although the ¹¹B resonance broadens in addition to shifting downfield as the molar fraction of HP-γ-CD is increased, the sharp nature of the Job plot speaks to the strength of the interaction.

The complex formed upon combination of Na₂B₁₂I₁₂ and HP-γ-CD is significantly more soluble than the complex with unfunctionalized γ-CD; no precipitation is observed upon combination of equivalent volumes of 200 mM Na₂B₁₂I₁₂ and 200 mM HP-γ-CD (affording a 100 mM solution of the complex).

Hemolysis Studies with Complexes of Na₂B₁₂I₁₂ and Substituted γ-Cyclodextrin

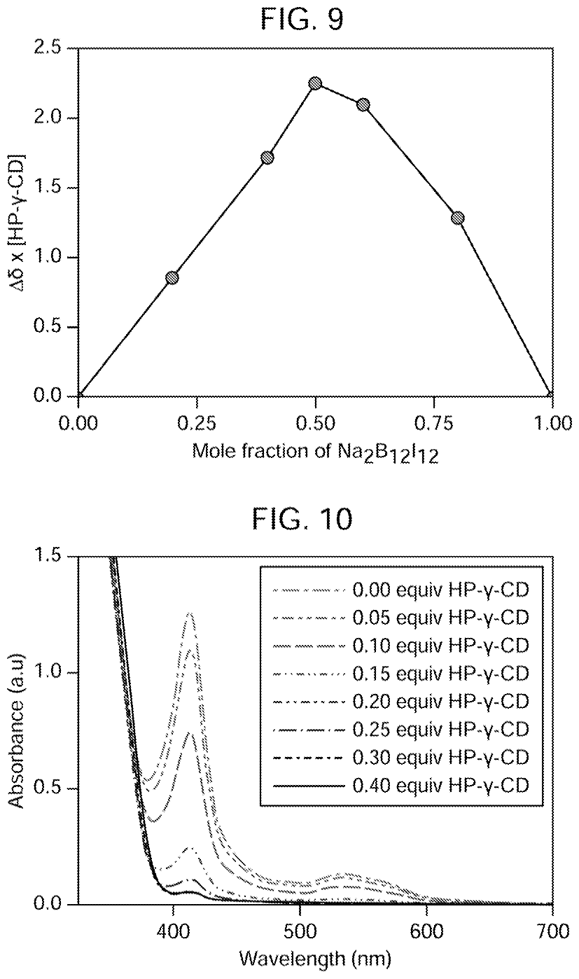

A Hb-release hemolysis assay was performed by suspending RBCs in solutions that featured a consistent concentration of Na₂B₁₂I₁₂ (100 mM) but a systematic increase in the concentration of HP-γ-CD. As was also demonstrated in FIG. 3, these experiments confirm that in the absence of cyclodextrin, 100 mM Na₂B₁₂I₁₂ results in complete hemolysis. Strikingly, the presence of even small amounts of 2-hydroxypropyl-γ-CD results in a drastic decrease in hemolysis (FIG. 10).

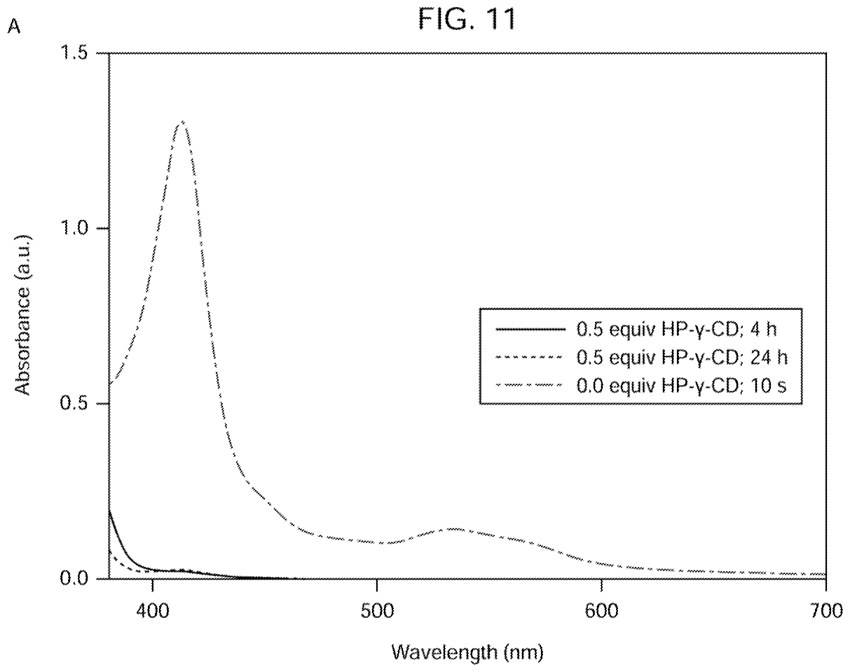

This attenuation of hemolysis increases with increasing HP-γ-CD concentration until it falls to near-baseline levels upon the addition of 0.4 equiv (FIG. 10). This protection was also maintained over periods of time reflective of the time over which an XCA would remain in circulation: RBC incubation with 100 mM Na₂B₁₂I₁₂ and 50 mM HP-γ-CD (0.5 equiv) for either 4 h or 24 h resulted in no hemolysis (FIG. 11).

### CONCLUSIONS

These results show that Na₂B₁₂I₁₂ induces rapid hemolysis at the concentrations used in medical imaging. This disruption of cell structure may be the origin of the toxicity of Na₂B₁₂I₂ and likely arises from the superchaotropic nature of the B₁₂I₁₂²⁻ anions. These results are corroborated by the ability of T-CD, which binds to B₁₂I₁₂²⁻ because of its superchaotropic nature, to inhibit hemolysis. X-ray crystallography suggests that, in the solid state, B₁₂I₁₂²⁻ may interact with T-CD in the same manner as previously reported for B₁₂Br₁₂²: formation of a 2:1 γ-CD:borate complex. Solution-phase data support the formation of a 1:1 complex in solution. The complex formed upon addition of Na₂B₁₂I₁₂ to T-CD exhibits water solubility that is too low to observe any hemolysis-protective effect. The derivatized cyclic oligosaccharide HP-T-CD forms a 1:1 complex with B₁₂I₁₂²⁻ that is much more water-soluble. The protective effect of HP-T-CD can be observed in hemolysis assays, where it can prevent cell destruction when added at substoichiometric levels. The 100 mM solutions of Na₂B₁₂I₂ with 0.4 equiv of IP-γ-CD feature an iodine concentration of 153 mg iodine mL⁻¹.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a feature in the claim only when the exact phrase “means for” or the exact phrase “step for” is recited at the beginning of such feature in the claim; if such exact phrase is not used in a feature in the claim, then 35 U.S.C. § 112(f) or 35 U.S.C. § 112(6) is not invoked.

What is claimed is:

1. A complex comprising:
a substituted cyclodextrin; and
a closo-dodecaiodododecaborate or a salt thereof.

2. The complex according to claim 1, wherein the substituted cyclodextrin is substituted with a hydroxy-substituted alkyl.

3. The complex according to claim 2, wherein the substituted cyclodextrin comprises 2-hydroxypropyl cyclodextrin.

4. The complex according to any one of claims 1-3, wherein the substituted cyclodextrin is a gamma cyclodextrin (γ-CD).

5. The complex according to any one of claims 1-4, wherein the substituted cyclodextrin is substituted at the 2 position, the 3 position, the 6 position or a combination thereof.

6. The complex according to claim 5, wherein the cyclodextrin is a compound of formula CD-I: 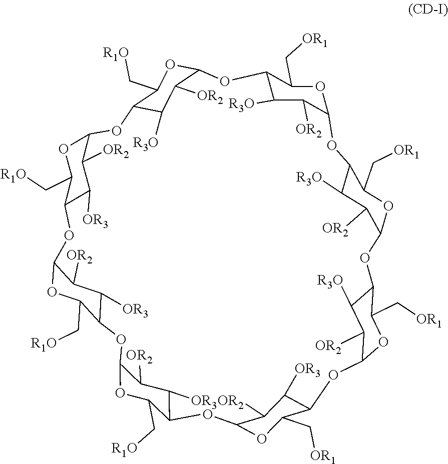
wherein each R₁, R₂ and R₃ are independently selected from substituted alkyl and substituted heteroalkyl.

7. The complex according to claim 6, wherein the substituted cyclodextrin is a compound of formula CD-101:

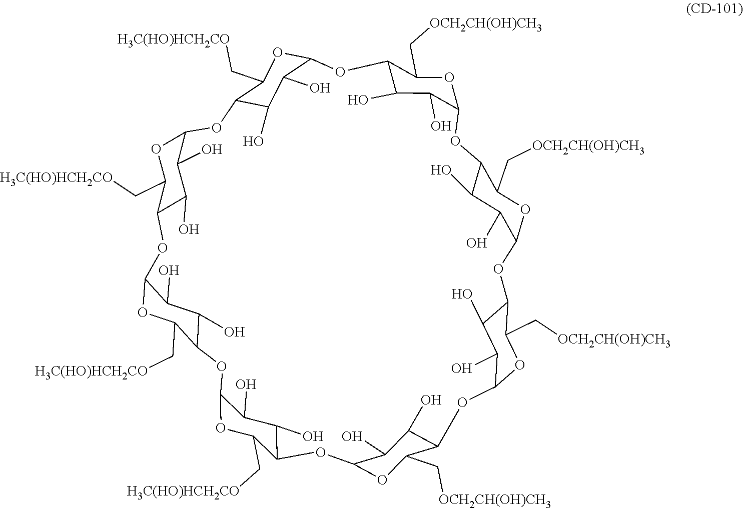

8. The complex according to any one of claims 1-7, wherein the complex comprises a salt of closo-dodecaiodododecaborate.

9. The complex according to any one of claims 1-8, wherein the complex comprises a 1:1 ratio of the substituted cyclodextrin to closo-dodecaiodododecaborate or a 2:1 ratio of the substituted cyclodextrin to closo-dodecaiodododecaborate.

10. A composition comprising:
a complex according to any one of claims 1-9 comprising:
a substituted cyclodextrin; and
a closo-dodecaiodododecaborate salt; and
a pharmaceutically acceptable excipient.

11. A method comprising administering a complex according to any one of claims 1-9 or a composition according to claim 10 to a subject.

12. The method according to claim 11, wherein the method comprises imaging the subject with a source of X-ray radiation.

13. The method according to any one of claims 11-12, wherein the composition is administered orally to the subject, administered to the subject by injection or administered intravenously to the subject.

14. The method according to any one of claims 11-13, wherein the complex is administered in an amount sufficient that does not cause hemolysis in the subject.

15. The method according to any one of claims 11-14, wherein the method further comprises generating an X-ray image of the subject.
